(19) United States
(12) Patent Application Publication
(10) Pub. No.: US 2017/0247455 A1
(43) Pub. Date: Aug. 31, 2017

(54) TREATMENT OF CANCER USING A COMBINATION OF AN ANTI-PD-1 ANTIBODY AND AN ANTI-CD137 ANTIBODY

(71) Applicant: BRISTOL-MYERS SQUIBB COMPANY, Princeton, NJ (US)

(72) Inventors: Maria JURE-KUNKEL, Plainsboro, NJ (US); David M. Berman, Potomac, MD (US); Alan J. Korman, Piedmont, CA (US); Mark J. Selby, San Francisco, CA (US); Suba Krishnan, New York, NY (US)

(21) Appl. No.: 15/505,299

(22) PCT Filed: Aug. 21, 2015
(86) PCT No.: PCT/US2015/046207

§ 371 Date: Feb. 21, 2017

Related U.S. Application Data
(60) Provisional application No. 62/040,704, filed on Aug. 22, 2014.

(51) Int. Cl. C07K 16/28; C07K 16/30

(52) U.S. Cl. CPC C07K 16/2818; C07K 16/2878; C07K 16/3061; A61K 2039/545

(57) ABSTRACT

This disclosure provides a method for treating a subject afflicted with a cancer, which method comprises administering to the subject therapeutically effective amounts of: (a) an antibody or an antigen-binding portion thereof that specifically binds to PD-1; and (b) an antibody or an antigen-binding portion thereof that specifically binds to CD137.

This application claims benefit to provisional application U.S. Ser. No. 62/040,704 filed Aug. 22, 2014, under 35 U.S.C. §119(e). The entire teachings of the referenced application are incorporated herein by reference.

### FIELD OF THE INVENTION

This invention relates to methods for treating cancer in a subject comprising administering to the subject a combination of an anti-Programmed Death-1 (PD-1) antibody and an anti-CD137 (also known as 4-1BB) antibody.

### BACKGROUND OF THE INVENTION

In recent years there has been a growing shift in the understanding of the biology and immunology of cancer, with the recognition that the immune system provides built-in defense mechanisms against not only infectious agents but cancers as well. It is increasingly appreciated that cancers are recognized by the immune system, and under some circumstances, the immune system may control or even eliminate tumors. However, the immune system also exerts a major effort to avoid immune over-activation, which could harm healthy tissues. Cancer takes advantage of this ability to hide from the immune system by exploiting a series of immune escape mechanisms that were developed to avoid autoimmunity. Among these mechanisms are the hijacking of immune-cell-intrinsic checkpoints that are induced on T-cell activation (Pardoll et al., Nat. Rev. Cancer, 12:252-264 (2012)). A novel approach in immunotherapy of cancer has been to counteract these resistance mechanisms, activating and allowing the endogenous immune system to reject tumors. Blockade of one of these checkpoints, cytotoxic T-lymphocyte-associated antigen 4 (CTLA-4), provided the first clinical evidence of improvement in overall survival for the treatment of patients with metastatic melanoma (Hodi et al., N. Engl. J. Med., 363:711-723 (2010) and 363:1290 (2010) (erratum); Robert et al., N. Engl. J. Med., 364:2517-2526 (2011)).

The Programmed Death 1 receptor (PD-1) is another key checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to down-regulate T cell activation and cytokine secretion upon binding to PD-1. Inhibition of the PD-1/PD-L1 interaction mediates potent antitumor activity in preclinical models (U.S. Pat. Nos. 8,008,449 and 7,943,743), and the use of antibody inhibitors of the PD-1/PD-L1 interaction for treating cancer has entered clinical trials (Brahmer et al., J. Clin. Oncol., 28:3167-3175 (2010); Topalian et al., N. Engl. J. Med., 366:2443-2454 (2012); Topalian et al., J. Clin. Oncol., 32(10):1020-1030 (2014); Hamid et al., N. Engl. J. Med., 369:134-144 (2013); Brahmer et al., N. Engl. J. Med., 366:2455-2465 (2012); Flies et al., Yale J. Biol. Med., 84:409-421 (2011); Pardoll, Nat. Rev. Cancer, 12:252-264 (2012); Hamid et al., Expert Opin. Biol. Ther., 13(6):847-861 (2013)).

CD137 is a T-cell costimulatory receptor induced on TCR activation (Nam et al., Curr. Cancer Drug Targets, 5:357-363 (2005); Watts et al., Annu. Rev. Immunol., 23:23-68 (2005)). In addition to its expression on activated CD4+ and CD8+ T cells, CD137 is also expressed on CD4+CD25+ regulatory T cells, activated natural killer (NK) and NK-T cells, monocytes, neutrophils, and dendritic cells. Its natural ligand, CD137L, has been described on antigen-presenting cells including B cells, monocyte/macrophages, and dendritic cells (Watts et al., Annu. Rev. Immunol., 23:23-68 (2005)). On interaction with its ligand, CD137 leads to increased TCR-induced T-cell proliferation, cytokine production, functional maturation, and prolonged CD8+ T-cell survival (Nam et al., supra; Watts et al., supra). Use of antibodies to activate the CD137 pathway for treating cancer has entered clinical trials (Li et al., Clin. Pharmacol., 5(Suppl 1):47-53 (2013); Sznol et al., J. Clin. Oncol. (Meeting Abstracts), 26(Suppl 15):3007 (2008)).

In spite of the promising antitumor efficacy of several monoclonal antibodies, many tumors are refractory to treatment with a single antibody (Wilcox et al., J. Clin. Invest., 109:651-659 (2002); Verbrugge et al., Cancer Res., 72:3163-3174 (2012)), and combinations of two or more antibodies may be needed. It is thus an object of the present invention to provide improved methods for treating cancer patients with a combination of different monoclonal antibodies.

### SUMMARY OF THE INVENTION

In certain embodiments, the present invention provides a method for treating a cancer in a human patient, such as solid tumors (e.g., advanced refractory solid tumors) or B cell lymphoma, comprising administering to the patient a combination of an anti-CD137 antibody and an anti-PD-1 antibody, wherein the combination is administered (or is for administration) according to a particular clinical dosage regimen (i.e., at a particular dose amount and according to a specific dosing schedule). In one embodiment, the human patient suffers from a cancer selected from melanoma (MEL), prostate cancer (PC), non-small cell lung cancer (NSCLC), colorectal cancer (CRC), head and neck squamous cell carcinoma (SCCHN), renal cell carcinoma (RCC), gastric carcinoma (GC), glioblastoma (GBM), and Non-Hodgkin's Lymphoma (NHL). For example, the anti-CD137 antibody is urelumab. For example, the anti-PD-1 antibody is nivolumab. Optionally, the anti-CD137 antibody is PF-05082566. Optionally, the anti-PD-1 antibody is pembrolizumab.

In certain embodiments, the subject has been pre-treated for the cancer. In other embodiments, the cancer is an advanced, metastatic and/or refractory cancer. In preferred embodiments, the administration of the combination of the anti-PD-1 antibody and the anti-CD137 antibody induces a durable clinical response in the patient.

An exemplary anti-CD137 antibody is BMS-663513 (i.e., urelumab) comprising heavy and light chains comprising the sequences shown in SEQ ID NOs: 1 and 2, respectively, or antigen binding fragments and variants thereof. In other embodiments, the antibody comprises the heavy and light chain complementarity determining regions (CDRs) or variable regions (VRs) of BMS-663513. Accordingly, in one embodiment, the antibody comprises CDR1, CDR2, and CDR3 domains of the heavy chain variable (VH) region of BMS-663513 having the sequence shown in SEQ ID NO: 3, and CDR1, CDR2 and CDR3 domains of the light chain variable (VL) region of BMS-663513 having the sequence shown in SEQ ID NO: 4. In another embodiment, the antibody comprises CDR1, CDR2 and CDR3 heavy chain sequences set forth in SEQ ID NOs: 5, 6, and 7, respectively, and CDR1, CDR2 and CDR3 light chain sequences as set forth in SEQ ID NOs: 8, 9, and 10, respectively. In another embodiment, the antibody has VH and/or VL regions comprising the amino acid sequences set forth in SEQ ID NO: 3 and/or SEQ ID NO: 4, respectively. In another embodiment, the antibody competes for binding with, and/or binds to the same epitope on CD137 as, the above-mentioned antibodies. In another embodiment, the antibody has at least about 90% variable region amino acid sequence identity with the above-mentioned antibodies (e.g., at least about 90%, 95% or 99% variable region identity with SEQ ID NO: 3 or SEQ ID NO: 4). Optionally, the anti-CD-137 antibody may be a chimeric, humanized or human monoclonal antibody or a portion thereof. Optionally, the anti-CD137 antibody comprises a heavy chain constant region which is of a human IgG1, IgG2 and IgG4 and variants thereof.

An exemplary anti-PD-1 antibody is nivolumab (also referred to as “5C4” in PCT Publication No. WO 2006/121168; and known as BMS-936558, MDX-1106 and ONO-4538) comprising heavy and light chains comprising the sequences shown in SEQ ID NOs: 11 and 12, respectively, or antigen binding fragments and variants thereof. In other embodiments, the antibody comprises the heavy and light chain CDRs or VRs of BMS-936558. Accordingly, in one embodiment, the antibody comprises CDR1, CDR2, and CDR3 domains of the VH region of BMS-936558 having the sequence shown in SEQ ID NO: 13, and CDR1, CDR2 and CDR3 domains of the VL region of BMS-936558 having the sequence shown in SEQ ID NO: 14. In another embodiment, the antibody comprises heavy chain CDR1, CDR2 and CDR3 domains comprising the sequences set forth in SEQ ID NOs: 15, 16, and 17, respectively, and light chain CDR1, CDR2 and CDR3 domains comprising the sequences set forth in SEQ ID NOs: 18, 19, and 20, respectively. In another embodiment, the antibody comprises VH and/or VL regions comprising the amino acid sequences set forth in SEQ ID NO: 13 and/or SEQ ID NO: 14, respectively. In another embodiment, the antibody competes for binding with, and/or binds to the same epitope on PD-1 as, the above-mentioned antibodies. In another embodiment, the antibody has at least about 90% variable region amino acid sequence identity with the above-mentioned antibodies (e.g., at least about 90%, 95% or 99% variable region identity with SEQ ID NO: 13 or SEQ ID NO: 14). Optionally, the anti-PD-1 antibody may be a chimeric, humanized or human monoclonal antibody or a portion thereof. Optionally, the anti-PD-1 antibody comprises a heavy chain constant region which is of a human IgG1, IgG2 and IgG4 and variants thereof.

In certain embodiments, the anti-PD-1 antibody is administered at a dose ranging from 0.1 to 10.0 mg/kg body weight once every 2, 3 or 4 weeks (e.g., a dose of 1 or 3 mg/kg body weight once every 2 weeks). In certain embodiments, the anti-CD137 antibody is administered at a dose ranging from 1 to 10 mg once every 4 or 8 weeks (e.g., a dose of 3 or 8 mg once every 4 weeks). Optionally, the dose of the anti-CD137 antibody and/or the anti-PD-1 antibody is calculated per mg/kg body weight (e.g., a dose of about 0.03-1 mg/kg, of about 0.03 mg/kg, of about 0.1 mg/kg, or about 0.3 mg/kg). Optionally, the dose of the anti-CD137 antibody and/or the anti-PD-1 antibody is a flat-fixed dose (e.g., a dose of about 3 mg-8 mg, of about 3 mg, or about 8 mg). In one embodiment, dosage regimens are adjusted to provide the optimum desired response (e.g., an effective response).

In certain embodiments, the method comprises at least one treatment cycle, wherein the cycle is a period of eight weeks. For example, the anti-PD-1 antibody is administered on Days 1, 15, 29, and 43 of each cycle. For example, the anti-CD137 antibody is administered on Days 1 and 29 of each cycle or Day 1 of each cycle. In one embodiment, the anti-PD-1 antibody is administered prior to administration of the anti-CD137 antibody. In another embodiment, the anti-PD-1 antibody is administered after administration of the anti-CD137 antibody. Optionally, the treatment consists of up to 12 cycles (e.g., at least 1, 2, 3, 4, 5, 6, 7, 8, 9, 10, 11, or 12 treatment cycles of eight weeks). In one specific embodiment, the anti-PD-1 antibody and the anti-CD137 antibody are administered during the first three cycles (cycles 1-3).

In one embodiment, the anti-PD-1 antibody and the anti-CD137 antibody are administered as a first (“front”) line of treatment (e.g., the initial or first treatment). In another embodiment, the anti-PD-1 antibody and the anti-CD137 antibody are administered as a second line of treatment (e.g., after initial treatment with the same or a different therapeutic, including after relapse and/or where the first treatment has failed). The anti-CD137 and anti-PD-1 antibodies can be administered to a subject by any suitable means. In one embodiment, the antibodies are formulated for intravenous administration. In another embodiment, the antibodies are administered simultaneously (e.g., formulated together in a single formulation or concurrently as separate formulations). Alternatively, in another embodiment, the antibodies are administered sequentially (e.g., as separate formulations).

In certain specific embodiments, the anti-CD137 antibody and anti-PD-1 antibody are administered at one of the following dosing regimens:

(a) 3 mg of the anti-CD137 antibody every 4 weeks and 3 mg/kg of the anti-PD-1 antibody every 2 weeks;

(b) 8 mg of the anti-CD137 antibody every 4 weeks and 3 mg/kg of the anti-PD-1 antibody every 2 weeks;

(c) 8 mg of the anti-CD137 antibody every 8 weeks and 3 mg/kg of the anti-PD-1 antibody every 2 weeks;

(d) 8 mg of the anti-CD137 antibody every 8 weeks and 1 mg/kg of the anti-PD-1 antibody every 2 weeks;

(e) 3 mg of the anti-CD137 antibody every 4 weeks and 1 mg/kg of the anti-PD-1 antibody every 2 weeks; and

(f) 3 mg of the anti-CD137 antibody every 8 weeks and 3 mg/kg of the anti-PD-1 antibody every 2 weeks.

In certain specific embodiments, the methods of the present invention comprise (a) an induction phase, wherein the anti-PD-1 and anti-CD137 antibodies are administered; followed by (b) a maintenance phase, wherein no anti-CD137 antibody is administered and the anti-PD-1 antibody is repeatedly administered at a dosage ranging from 0.1 to 10 mg/kg body weight every 2, 3 or 4 weeks (e.g., at a dose of 1 or 3 mg/kg body weight once every 2 weeks). Optionally, the induction phase may consist of at least 1, 2 or 3 cycles (e.g., 8 weeks/cycle). Optionally, the maintenance phase may consist of at least 1, 2, 3, 4, 5, 6, 7, 8, 9, or 10 cycles, or continue as long as clinical benefit is observed or until unmanageable toxicity or disease progression occurs.

The efficacy of the treatment methods provided herein can be assessed using any suitable means. In one embodiment, the treatment produces at least one therapeutic effect selected from the group consisting of reduction in size of a tumor, reduction in number of metastatic lesions over time, complete response, partial response, and stable disease.

The disclosure also provides a kit for treating a subject afflicted with a cancer, the kit comprising: (a) a dosage ranging from 0.1 to 10 mg/kg body weight of an anti-PD-1 antibody or an antigen-binding portion thereof; (b) a dosage ranging from 1 to 10 mg of an anti-CD137 antibody or an antigen-binding portion thereof; and (c) instructions for using the anti-PD-1 antibody and the anti-CD137 antibody in a method of the present invention.

Other features and advantages of the instant invention will be apparent from the following detailed description and examples which should not be construed as limiting. The contents of all cited references, including scientific articles, newspaper reports, GENBANK® entries, patents and patent applications cited throughout this application are expressly incorporated herein by reference.

### BRIEF DESCRIPTION OF THE DRAWINGS

FIG. 1 shows a combination therapy with a mouse anti-CD137 antibody and a mouse anti-PD-1 antibody in an MC38 colon cancer mouse model.

FIG. 2 shows a combination therapy with a mouse anti-CD137 antibody and a mouse anti-PD-1 antibody in an M109 lung adenocarcinoma mouse model.

FIG. 3 is a schematic illustrating the study design of the clinical trial CA186107 (urelumab in combination with nivolumab).

FIG. 4 shows the heavy and light chain sequences of anti-CD137 antibody, urelumab. The variable region of each chain is designated by underlining, the constant region of each chain is designated in bold, and the respective CDR regions (CDR1, CDR2, and CDR3) are designated in blocked text.

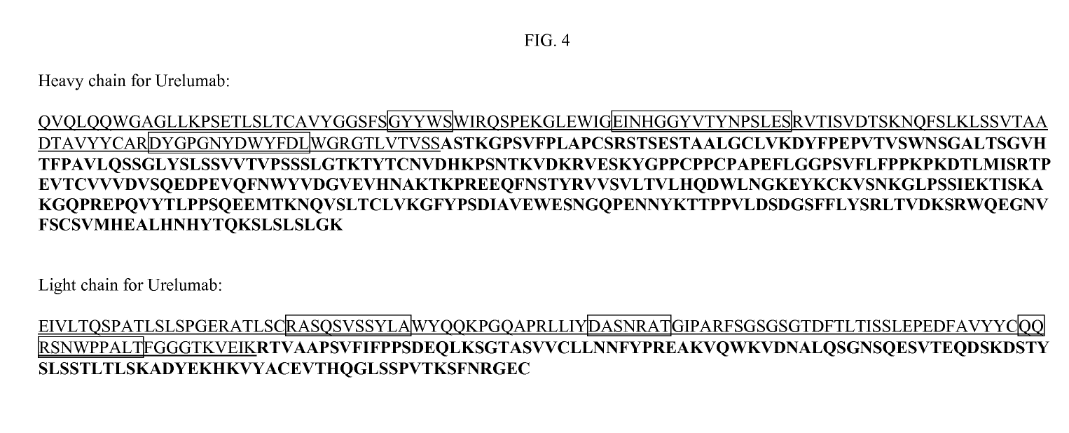

FIG. 5 shows the heavy and light chain sequences of anti-PD-1 antibody, nivolumab. The variable region of each chain is designated by underlining, the constant region of each chain is designated in bold, and the respective CDR regions (CDR1, CDR2, and CDR3) are designated in blocked text.

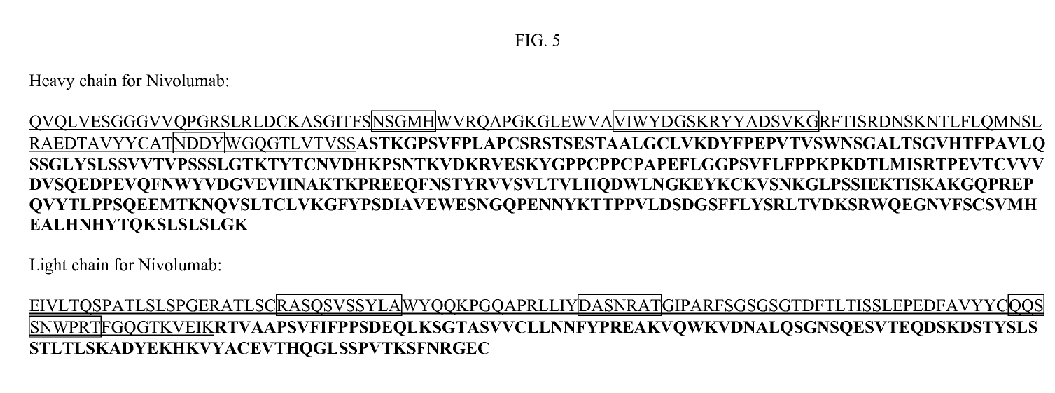

FIG. 6 shows a summary of activating/inhibitor ratios (A/I) for model antibodies containing different immunoglobulin isotypes based upon their affinity to inhibiting Fc receptors and activating Fc receptors as determined by Nimmerjahn et al. (Science, 310 (Dec. 2, 2005)). The ratios are useful for determining whether an antibody may have depleting capability which may be important for predicting in vivo depleting activity of an antibody in humans.

FIG. 7 shows tumor volumes of mice that were administered monotherapy with a mouse IgG control antibody after they were injected with MC38 colon cancer cells. As shown, none of the mice (0 out of 12) achieved a tumor free (TF) response.

FIG. 8 shows tumor volumes of mice that were administered monotherapy with a chimeric anti-mouse CD137 antibody containing a G1 isotype after the mice were injected with MC38 colon cancer cells. As shown, eleven of the mice (11 out of 12) achieved a tumor-free (TF) response.

FIG. 9 shows tumor volumes of mice that were administered monotherapy with an anti-mouse CD137 antibody containing a g1 isotype with a D265A mutation after the mice were injected with MC38 colon cancer cells. As shown, none of the mice (0 out of 12) achieved a tumor-free (TF) response.

FIG. 10 shows tumor volumes of mice that were administered monotherapy with an anti-mouse CD137 antibody containing a g2b isotype after the mice were injected with MC38 colon cancer cells. As shown, seven of the mice (7 out of 12) achieved a tumor-free (TF) response.

FIG. 11 shows tumor volumes of mice that were administered monotherapy with an anti-mouse CD137 antibody containing a g2a isotype after the mice were injected with MC38 colon cancer cells. As shown, six of the mice (6 out of 12) achieved a tumor-free (TF) response.

FIG. 12 shows tumor volumes of mice that were administered monotherapy with a chimeric anti-mouse PD-1 antibody after the mice were injected with MC38 colon cancer cells. As shown, two of the mice (2 out of 12) achieved a tumor-free (TF) response.

FIG. 13 shows tumor volumes of mice that were administered combination therapy with an anti-mouse PD-1 antibody in addition to mouse anti-CD137 containing a g1 isotype after the mice were injected with MC38 colon cancer cells. As shown, eleven of the mice (11 out of 12) achieved a tumor-free (TF) response.

FIG. 14 shows tumor volumes of mice that were administered combination therapy with an anti-mouse PD-1 antibody in addition to mouse anti-CD137 containing an IgG1 isotype with a D265A mutation (this mutation eliminates binding to Fc receptors) after the mice were injected with MC38 colon cancer cells. As shown, five of the mice (5 out of 12) achieved a tumor-free (TF) response.

FIG. 15 shows tumor volumes of mice that were administered combination therapy with an anti-mouse PD-1 antibody in addition to mouse anti-CD137 containing a g2b isotype after the mice were injected with MC38 colon cancer cells. As shown, eleven of the mice (11 out of 12) achieved a tumor free (TF) response.

FIG. 16 shows a summary of the mean tumor volumes of mice that were administered monotherapy with an anti-mouse PD1 antibody or an anti-mouse CD137 containing various isotypes after the mice were injected with MC38 colon cancer cells. As shown, anti-CD137 antibodies containing the mouse g2b isotype achieved the best response, followed by g1, followed by g2a.

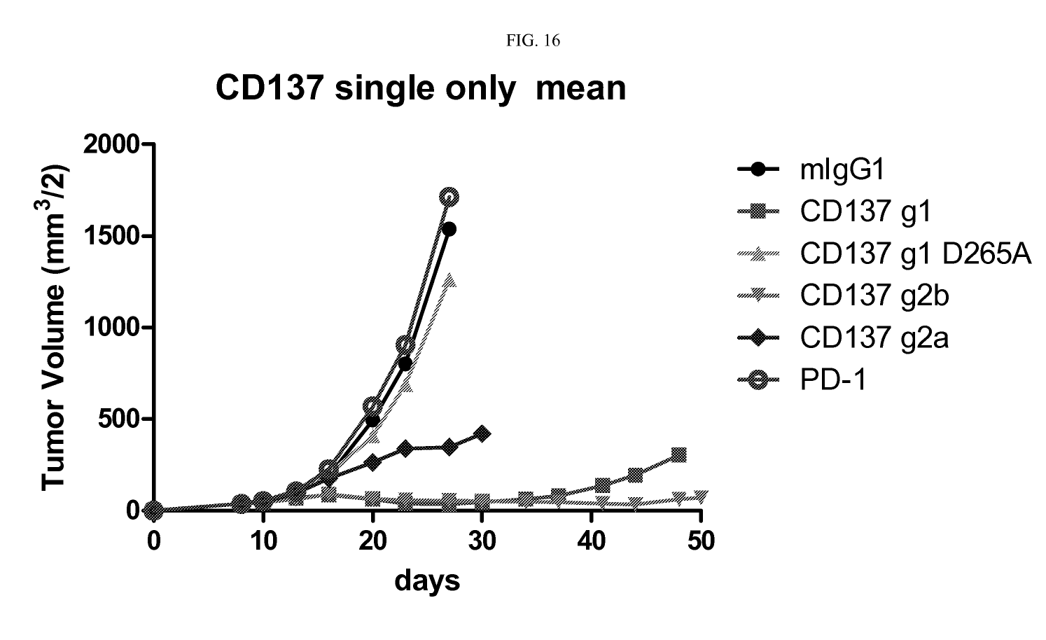

FIG. 17 shows a summary of the median tumor volumes of mice that were administered monotherapy with an anti-mouse PD1 antibody or an anti-mouse CD137 containing various isotypes after the mice were injected with MC38 colon cancer cells. As shown, anti-CD137 antibodies containing the mouse g2b, g1, and g2a isotypes achieved the best responses.

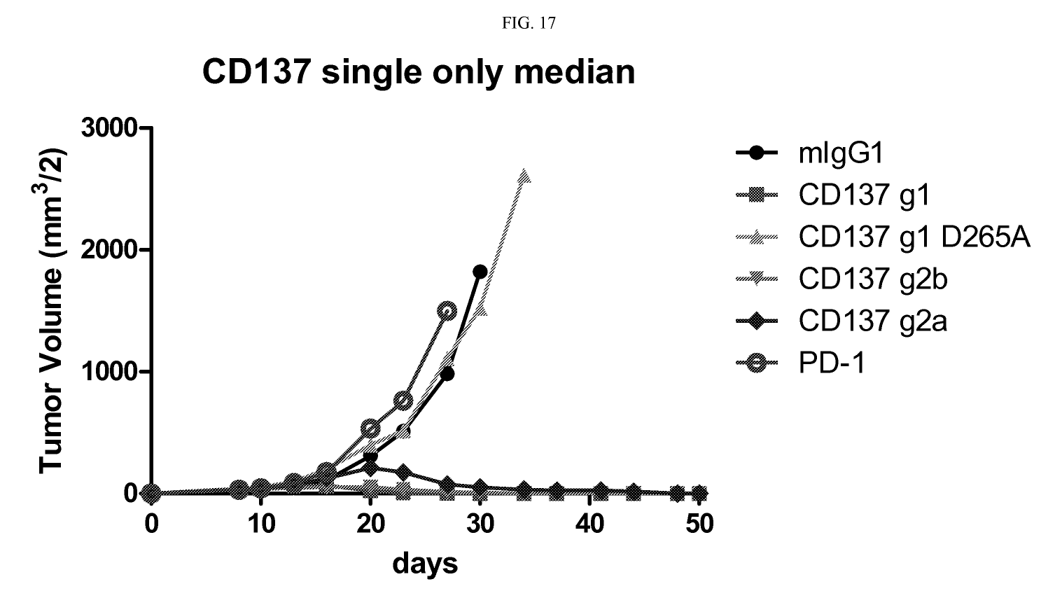

FIG. 18 shows a summary of the mean tumor volumes of mice that were administered combination therapy with an anti-mouse PD1 antibody and an anti-mouse CD137 containing various isotypes after the mice were injected with MC38 colon cancer cells. As shown, the combination of anti-PD1 and anti-CD137 antibodies containing the g2b isotype achieved the best response, followed by g1.

FIG. 19 shows a summary of the mean tumor volumes of mice that were administered combination therapy with an anti-mouse PD1 antibody and the mouse anti-CD137 containing various isotypes after the mice were injected with MC38 colon cancer cells. As shown, the combination of anti-PD1 and anti-CD137 antibodies containing the mouse g2b and g1 isotypes achieved the best responses.

FIG. 20 shows a summary of overall survival for mice that were administered either monotherapy or combination therapy with an anti-mouse anti-PD1 antibody an anti-mouse CD137 containing various isotypes after the mice were injected with MC38 colon cancer cells. As shown, the combination of anti-PD1 and anti-CD137 antibodies containing the g2b and g1 isotypes achieved the best responses.

### DETAILED DESCRIPTION OF THE INVENTION

The present invention relates to methods for treating a cancer patient comprising administering to the patient a combination of an anti-PD-1 antibody and an anti-CD137 antibody.

An anti-PD-1 monoclonal antibody (e.g., nivolumab) removes T cell inhibition and has demonstrated single agent activity in early phase clinical studies with a tolerable safety profile. Treatment with an anti-CD137 monoclonal antibody (e.g., urelumab, a non-blocking T cell agonist antibody) has demonstrated single agent activity in early clinical studies with a tolerable safety profile at doses<0.3 mg/kg. Although treatment with single agent immunotherapies has seen recent advances and continues to generate encouraging data in multiple tumor types, many tumors are refractory to treatment with a single antibody, and combinations of two or more mAbs may provide greater anti-tumor synergy with more durable responses.

The present invention is based at least in part on data from preclinical studies conducted in animal tumor models. The results demonstrated that the combination of an anti-CD137 antibody and an anti-PD-1 antibody showed synergy in terms of greater efficacy than the anti-PD-1 antibody or the anti-CD137 antibody alone.

### Definitions

In order that the present disclosure may be more readily understood, certain terms are first defined. As used in this application, except as otherwise expressly provided herein, each of the following terms shall have the meaning set forth below. Additional definitions are set forth throughout the application.

“Administering” refers to the physical introduction of a composition comprising a therapeutic agent to a subject, using any of the various methods and delivery systems known to those skilled in the art. Preferred routes of administration for the anti-PD-1 antibody and/or the anti-CD137 antibody include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. The TKI is typically administered via a non-parenteral route, preferably orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. For example, an adverse event may be associated with activation of the immune system or expansion of immune system cells (e.g., T cells) in response to a treatment. A medical treatment may have one or more associated AEs and each AE may have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

An “isolated antibody” refers to an Ab that is substantially free of other Abs having different antigenic specificities (e.g., an isolated Ab that binds specifically to PD-1 is substantially free of Abs that bind specifically to antigens other than PD-1). Moreover, an isolated Ab may be substantially free of other cellular material and/or chemicals.

The term “monoclonal antibody” (“mAb”) refers to a non-naturally occurring preparation of Ab molecules of single molecular composition, i.e., Ab molecules whose primary sequences are essentially identical, and which exhibits a single binding specificity and affinity for a particular epitope. A mAb is an example of an isolated Ab. MAbs may be produced by hybridoma, recombinant, transgenic or other techniques known to those skilled in the art.

A “human” antibody (HuMAb) refers to an Ab having variable regions in which both the framework and CDR regions are derived from human germline immunoglobulin sequences. Furthermore, if the Ab contains a constant region, the constant region also is derived from human germline immunoglobulin sequences. The human Abs of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include Abs in which CDR sequences derived from the germline of another mammalian species, such as a mouse, have been grafted onto human framework sequences. The terms “human” Abs and “fully human” Abs and are used synonymously.

A “humanized antibody” refers to an Ab in which some, most or all of the amino acids outside the CDR domains of a non-human Ab are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the Ab to bind to a particular antigen. A “humanized” Ab retains an antigenic specificity similar to that of the original Ab.

A “chimeric antibody” refers to an Ab in which the variable regions are derived from one species and the constant regions are derived from another species, such as an Ab in which the variable regions are derived from a mouse Ab and the constant regions are derived from a human Ab.

An “anti-antigen” Ab refers to an Ab that binds specifically to the antigen. For example, an anti-PD-1 Ab binds specifically to PD-1 and an anti-CD137 Ab binds specifically to CD137.

An “antigen-binding portion” of an Ab (also called an “antigen-binding fragment”) refers to one or more fragments of an Ab that retain the ability to bind specifically to the antigen bound by the whole Ab.

A “cancer” refers a broad group of various diseases characterized by the uncontrolled growth of abnormal cells in the body. Unregulated cell division and growth divide and grow results in the formation of malignant tumors that invade neighboring tissues and may also metastasize to distant parts of the body through the lymphatic system or bloodstream. The terms, “cancer”, “tumor”, and “neoplasm”, are used interchangeably herein.

“CD137”, also referred to as 4-1BB or TNFRSF9, refers to a TNF superfamily Type 1 membrane glycoprotein receptor, which is expressed on the surface of lymphoid organs and can be detected on activated T cells (CD4+ and CD8+), activated NK cells, natural killer T (NKT) cells, regulatory T cells, activated thymocytes, intraepithelial lymphocytes and eosinophils. The natural ligand for CD137 is designated as CD137L, a TNF superfamily Type II membrane glycoprotein. The term “CD137” as used herein includes human CD137 (hCD137), variants, isoforms, and species homologs of hCD137, and analogs having at least one common epitope with hCD137. The complete hCD137 cDNA and protein sequences can be found under GENBANK® Accession Nos. NM_001561 and NP_001552, respectively.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GENBANK® Accession No. U64863.

“Programmed Death Ligand-1 (PD-L1)” is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GENBANK® Accession No. Q9NZQ7.

A “subject” includes any human or nonhuman animal. The term “nonhuman animal” includes, but is not limited to, vertebrates such as nonhuman primates, sheep, dogs, and rodents such as mice, rats and guinea pigs. In preferred embodiments, the subject is a human. The terms, “subject” and “patient” are used interchangeably herein.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject. In preferred embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount of an anti-cancer agent preferably inhibits cell growth or tumor growth by at least about 20%, more preferably by at least about 40%, even more preferably by at least about 60%, and still more preferably by at least about 80% relative to untreated subjects. In other preferred embodiments of the invention, tumor regression may be observed and continue for a period of at least about 20 days, more preferably at least about 40 days, or even more preferably at least about 60 days. Notwithstanding these ultimate measurements of therapeutic effectiveness, evaluation of immunotherapeutic drugs must also make allowance for “immune-related” response patterns.

An “immune-related” response pattern refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents may require long-term monitoring of the effects of these agents on the target disease.

A therapeutically effective amount of a drug includes a “prophylactically effective amount”, which is any amount of the drug that, when administered alone or in combination with an anti-neoplastic agent to a subject at risk of developing a cancer (e.g., a subject having a pre-malignant condition) or of suffering a recurrence of cancer, inhibits the development or recurrence of the cancer. In preferred embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the invention are described in further detail in the following subsections.

### Anti-PD-1 Antibodies

HuMAbs that bind specifically to PD-1 with high affinity have been disclosed in U.S. Pat. No. 8,008,449. Other anti-PD-1 mAbs have been described in, for example, U.S. Pat. Nos. 6,808,710, 7,488,802, 8,168,757 and 8,354,509, and PCT Publication No. WO 2012/145493. Each of the anti-PD-1 HuMAbs disclosed in U.S. Pat. No. 8,008,449 has been demonstrated to exhibit one or more of the following characteristics: (a) binds to human PD-1 with a K_{D} of 1×10⁻⁷ M or less, as determined by surface plasmon resonance using a BIACORE® biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates Ab responses; and (j) inhibits tumor cell growth in vivo. Anti-PD-1 Abs usable in the present invention include mAbs that bind specifically to human PD-1 and exhibit at least one, preferably at least five, of the preceding characteristics.

A preferred anti-PD-1 Ab is nivolumab (also referred to as BMS-936558). Nivolumab is a fully human IgG4 anti-PD-1 monoclonal antibody disclosed as 5C4 in PCT Publication No. WO 2006/121168. Nivolumab is known to augment cellular immune responses against tumors (Brahmer, J. R. et al., J. Clin. Oncol., 28:3167-3175 (2010)). Another anti-PD-1 Ab usable in the present methods is pembrolizumab (Hamid et al., N. Engl. J. Med., 369(2):134-144 (2013)).

Anti-PD-1 Abs usable in the disclosed methods also include isolated Abs that bind specifically to human PD-1 and cross-compete for binding to human PD-1 with nivolumab (see, e.g., U.S. Pat. No. 8,008,449; PCT Publication No. WO 2013/173223). The ability of Abs to cross-compete for binding to an antigen indicates that these Abs bind to the same epitope region of the antigen and sterically hinder the binding of other cross-competing Abs to that particular epitope region. These cross-competing Abs are expected to have functional properties very similar those of nivolumab by virtue of their binding to the same epitope region of PD-1. Cross-competing Abs can be readily identified based on their ability to cross-compete with nivolumab in standard PD-1 binding assays such as BIACORE® analysis, ELISA assays or flow cytometry (see, e.g., PCT Publication No. WO 2013/173223).

For administration to human subjects, these anti-PD-1 Abs are preferably chimeric Abs, or more preferably humanized or human Abs. Such chimeric, humanized or human mAbs can be prepared and isolated by methods well known in the art. Anti-PD-1 Abs usable in the methods of the disclosed invention also include antigen-binding portions of the above Abs. It has been amply demonstrated that the antigen-binding function of an Ab can be performed by fragments of a full-length Ab. Examples of binding fragments encompassed within the term “antigen-binding portion” of an Ab include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; and (iv) a Fv fragment consisting of the V_{L} and V_{H} domains of a single arm of an Ab. Anti-PD-1 antibodies (or VH and/or VL domains derived therefrom) suitable for use in the invention can be generated using methods well known in the art.

An exemplary anti-PD-1 antibody is nivolumab comprising heavy and light chains comprising the sequences shown in SEQ ID NOs: 11 and 12, respectively, or antigen binding fragments and variants thereof.

In other embodiments, the antibody has heavy and light chain CDRs or variable regions of nivolumab. Accordingly, in one embodiment, the antibody comprises CDR1, CDR2, and CDR3 domains of the VH of nivolumab having the sequence set forth in SEQ ID NO: 13, and CDR1, CDR2 and CDR3 domains of the VL of nivolumab having the sequence set forth in SEQ ID NO: 14. In another embodiment, the antibody comprises CDR1, CDR2 and CDR3 domains comprising the sequences set forth in SEQ ID NOs: 15, 16, and 17, respectively, and CDR1, CDR2 and CDR3 domains comprising the sequences set forth in SEQ ID NOs: 18, 19, and 20, respectively. In another embodiment, the antibody comprises VH and/or VL regions comprising the amino acid sequences set forth in SEQ ID NO: 13 and/or SEQ ID NO: 14, respectively. In another embodiment, the antibody competes for binding with and/or binds to the same epitope on PD-1 as the above-mentioned antibodies. In another embodiment, the antibody has at least about 90% variable region amino acid sequence identity with the above-mentioned antibodies (e.g., at least about 90%, 95% or 99% variable region identity with SEQ ID NO: 13 or SEQ ID NO: 14).

### Anti-CD137 Antibodies

Anti-CD137 antibodies of the instant invention specifically bind to human CD137. Preferably, the anti-CD137 antibodies are agonistic antibodies which activate the CD137 pathway. In certain embodiments, the anti-CD137 antibodies provide a strong costimulatory signal to T cells and NK cells, resulting in enhanced cytokine production (chiefly IFNγ), survival and proliferation. Preferably, the anti-CD137 antibodies enhance the function of antigen-specific T cells and mediate clinical antitumor activity by enhancing the host anti-tumor immune response. Anti-CD137 Abs usable in the disclosed methods include the antibodies disclosed in U.S. Publication No. 2005/0095244, the antibodies disclosed in issued U.S. Pat. No. 7,288,638 (such as 20H4.9-IgG4 [1007 or BMS-663513] or 20H4.9-IgG1 [BMS-663031]); the antibodies disclosed in issued U.S. Pat. No. 6,887,673 [4E9 or BMS-554271]; the antibodies disclosed in issued U.S. Pat. No. 7,214,493; the antibodies disclosed in issued U.S. Pat. No. 6,303,121; the antibodies disclosed in issued U.S. Pat. No. 6,569,997; the antibodies disclosed in issued U.S. Pat. No. 6,905,685; the antibodies disclosed in issued U.S. Pat. No. 6,355,476; the antibodies disclosed in issued U.S. Pat. No. 6,362,325 [1D8 or BMS-469492; 3H3 or BMS-469497; or 3E1]; the antibodies disclosed in issued U.S. Pat. No. 6,974,863 (such as 53A2); or the antibodies disclosed in issued U.S. Pat. No. 6,210,669 (such as 1D8, 3B8, or 3E1), and the CD137 agonistic antibodies described in U.S. Pat. Nos. 5,928,893, 6,303,121 and 6,569,997, the teachings of which are hereby incorporated by reference herein in their entirety, and in particular, those portions directly related to these antibodies. Antibodies that compete with any of these art-recognized antibodies for binding to CD137 also can be used.

A preferred anti-CD137 Ab is urelumab (also referred to as BMS-663513). Urelumab is a fully human IgG4 monoclonal antibody disclosed as antibody 1007 in U.S. Pat. No. 7,288,638. Urelumab is known to augment cellular immune responses against tumors (Melero, I. et al., Trends Pharmacol. Sci., 29(8):383-390 (2008)). Another anti-CD137 Ab usable in the present methods is PF-05082566 (Fisher et al., Cancer Immunol. Immunother., 61(10):1721-1733 (2012)).

Anti-CD137 Abs usable in the disclosed methods also include isolated Abs that bind specifically to human CD137 and cross-compete for binding to human CD137 with urelumab or bind to the same epitope region of human CD137 as urelumab. The ability of Abs to cross-compete for binding to an antigen indicates that these Abs bind to the same epitope region of the antigen and sterically hinder the binding of other cross-competing Abs to that particular epitope region. These cross-competing Abs are expected to have functional properties very similar those of urelumab by virtue of their binding to the same epitope region of CD137. Cross-competing Abs can be readily identified based on their ability to cross-compete with urelumab in standard CD137 binding assays such as BIACORE® analysis, ELISA assays or flow cytometry (e.g., U.S. Pat. No. 7,288,638).

For administration to human subjects, these anti-CD137 Abs are preferably chimeric Abs, or more preferably humanized or human Abs. Such chimeric, humanized or human mAbs can be prepared and isolated by methods well known in the art. Usable anti-CD137 Abs also include antigen-binding portions of the above Abs. It has been amply demonstrated that the antigen-binding function of an Ab can be performed by fragments of a full-length Ab. Examples of binding fragments encompassed within the term “antigen-binding portion” of an Ab include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; and (iv) a Fv fragment consisting of the V_{L} and V_{H} domains of a single arm of an Ab. Anti-CD137 antibodies (or VH and/or VL domains derived therefrom) suitable for use in the invention can be generated using methods well known in the art.

An exemplary anti-CD137 antibody is urelumab comprising heavy and light chains having the sequences shown in SEQ ID NOs: 1 and 2, respectively, or antigen binding fragments and variants thereof.

In other embodiments, the antibody has heavy and light chain CDRs or variable regions of urelumab. Accordingly, in one embodiment, the antibody comprises CDR1, CDR2, and CDR3 domains of the VH of urelumab having the sequence set forth in SEQ ID NO: 3, and CDR1, CDR2 and CDR3 domains of the VL of nivolumab having the sequence set forth in SEQ ID NO: 4. In another embodiment, the antibody comprises CDR1, CDR2 and CDR3 domains comprising the sequences set forth in SEQ ID NOs: 5, 6, and 7, respectively, and CDR1, CDR2 and CDR3 domains comprising the sequences set forth in SEQ ID NOs: 8, 9, and 10, respectively. In another embodiment, the antibody comprises VH and/or VL regions comprising the amino acid sequences set forth in SEQ ID NO: 3 and/or SEQ ID NO: 4, respectively. In another embodiment, the antibody competes for binding with and/or binds to the same epitope on CD137 as the above-mentioned antibodies. In another embodiment, the antibody has at least about 90% variable region amino acid sequence identity with the above-mentioned antibodies (e.g., at least about 90%, 95% or 99% variable region identity with SEQ ID NO: 3 or SEQ ID NO: 4).

### Pharmaceutical Compositions

Therapeutic agents (e.g., anti-PD-1 antibodies and/or anti-CD137 antibodies) of the present invention may be constituted in a composition, e.g., a pharmaceutical composition containing and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. “Pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions (e.g., comprising an anti-CD137 antibody and/or anti-PD-1 antibody). Preferably, the carrier for a composition containing an Ab is suitable for intravenous, intramuscular, subcutaneous, parenteral, spinal or epidermal administration (e.g., by injection or infusion). A pharmaceutical composition of the invention may include one or more pharmaceutically acceptable salts, anti-oxidant, aqueous and non-aqueous carriers, and/or adjuvants such as preservatives, wetting agents, emulsifying agents and dispersing agents.

Liquid compositions for parenteral administration can be formulated for administration by injection or continuous infusion. Routes of administration by injection or infusion include intravenous, intraperitoneal, intramuscular, intrathecal and subcutaneous. In one embodiment, the anti-CD137 antibody and/or the anti-PD-1 antibody are administered intravenously (e.g., in separate formulations or in the same formulation).

### Patient Populations

Provided herein are clinical methods for treating a cancer (e.g., a solid tumor or B cell lymphoma) in human patients using a combination of an anti-CD137 antibody and an anti-PD-1 antibody.

Examples of cancers that may be treated using the methods of the invention, include liver cancer, bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, and any combinations of said cancers. The present invention is also applicable to treatment of metastatic cancers.

In one embodiment, the human patient suffers from a cancer selected from melanoma, prostate cancer, non-small cell lung cancer, colorectal cancer, head and neck squamous cell carcinoma, renal cell carcinoma, gastric carcinoma, glioblastoma, and Non-Hodgkin's Lymphoma (NHL).

Patients can be tested or selected for one or more of the above described clinical attributes prior to, during or after treatment.

### Combination Therapies

Combination therapies provided herein involve administration of an anti-CD137 antibody and an anti-PD-1 antibody to treat subjects having a cancer (e.g., a solid tumor or a B cell lymphoma). In a particular embodiment, the anti-CD137 antibody is urelumab. In another embodiment, the anti-PD-1 antibody is nivolumab. In another embodiment, dosage regimens are adjusted to provide the optimum desired response (e.g., an effective response).

As used herein, adjunctive or combined administration (coadministration) includes simultaneous administration of the compounds in the same or different dosage form, or separate administration of the compounds (e.g., sequential administration). Thus, the anti-CD137 and anti-PD-1 antibodies can be simultaneously administered in a single formulation. Alternatively, the anti-CD137 and anti-PD-1 antibodies can be formulated for separate administration and are administered concurrently or sequentially.

For example, the anti-PD1 antibody can be administered first followed by (e.g., immediately followed by) the administration of the anti-CD137 antibody, or vice versa. In one embodiment, the anti-PD-1 antibody is administered prior to administration of the anti-CD137 antibody. In another embodiment, the anti-PD-1 antibody is administered after administration of the anti-CD137 antibody. In another embodiment, the anti-CD137 antibody and anti-PD-1 antibody are administered concurrently. Such concurrent or sequential administration preferably results in both antibodies being simultaneously present in treated patients.

In one embodiment, the dose of the anti-CD137 and/or anti-PD-1 antibody is calculated per body weight, e.g., mg/kg body weight. In another embodiment, the dose of the anti-CD137 and/or anti-PD-1 antibody is a flat-fixed dose. In another embodiment, the dose of the anti-CD137 and/or anti-PD-1 antibody is varied over time. For example, the anti-CD137 antibody and/or anti-PD-1 antibody may be initially administered at a high dose and may be lowered over time. In another embodiment, the anti-CD137 antibody and/or anti-PD-1 antibody is initially administered at a low dose and increased over time.

In another embodiment, the amount of the anti-CD137 and/or anti-PD-1 antibodies administered is constant for each dose. In another embodiment, the amount of antibody administered varies with each dose. For example, the maintenance (or follow-on) dose of the antibody can be higher or the same as the loading dose which is first administered. In another embodiment, the maintenance dose of the antibody can be lower or the same as the loading dose.

In another embodiment, the anti-PD-1 antibody and anti-CD137 antibody are administered as a first line of treatment (e.g., the initial or first treatment). In another embodiment, the anti-PD-1 antibody and anti-CD137 antibody are administered as a second line of treatment (e.g., after the initial or first treatment, including after relapse and/or where the first treatment has failed).

In certain embodiments, the combination of an anti-PD-1 Ab and an anti-CD137 Ab is administered intravenously to the subject in an induction phase, followed by a maintenance phase during which only the anti-PD-1 antibody is administered intravenously. To illustrate, the combination of nivolumab and urelumab is administered in the induction phase (e.g., cycles 1-3), followed by a maintenance phase (e.g., cycles 4-12) during which only nivolumab is administered to the subject.

Dosage and frequency vary depending on the half-life of the Ab in the subject. In general, human Abs show the longest half-life, followed by humanized Abs, chimeric Abs, and nonhuman Abs. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present invention can be administered via one or more routes of administration using one or more of a variety of methods well known in the art. As will be appreciated by the skilled artisan, the route and/or mode of administration will vary depending upon the desired results.

In one aspect, the invention features any of the aforementioned embodiments, wherein the anti-PD-1 antibody is replaced by, or combined with, an anti-PD-L1 or anti-PD-L2 antibody.

In certain embodiments, the anti-PD-1 antibody is administered at a dose ranging from about 0.1 to 10.0 mg/kg body weight once every 1, 2, 3 or 4 weeks. For example, the anti-PD-1 antibody is administered at a dose of 1 or 3 mg/kg body weight once every 2 weeks. In certain embodiments, the anti-CD137 antibody is administered at a dose ranging from about 1 to 10 mg (equivalent to about 0.01 to 0.1 mg/kg body weight) once every 4 or 8 weeks. For example, the anti-CD137 antibody is administered at a dose of 3 or 8 mg (equivalent to about 0.03 or 0.1 mg/kg body weight) once every 4 or 8 weeks.

In certain embodiments, the method comprises at least one treatment cycle (e.g., a treatment cycle consisting of eight weeks). To illustrate, in an eight-week cycle, the anti-PD-1 antibody is administered on Days 1, 15, 29, and 43. To illustrate, in an eight-week cycle, the anti-CD137 antibody is administered on Days 1 and 29 or Day 1. In one embodiment, the anti-PD-1 antibody is administered prior to administration of the anti-CD137 antibody. In another embodiment, the anti-PD-1 antibody is administered after administration of the anti-CD137 antibody. Optionally, the treatment cycle can be repeated up to 12 cycles (e.g., at least 1, 2, 3, 4, 5, 6, 7, 8, 9, 10, 11, or 12 cycles), or as long as a clinical benefit is observed or until there is a complete response, confirmed progressive disease or unmanageable toxicity.

In a specific embodiment, 4 doses of the anti-PD-1 antibody are administered per eight week cycle. In another specific embodiment, 1 or 2 doses of the anti-CD137 antibody are administered per eight week cycle.

In certain specific embodiments, the anti-CD137 antibody and anti-PD-1 antibody are administered at one of the following dosing regimens:

(a) 3 mg of the anti-CD137 antibody every 4 weeks and 3 mg/kg of the anti-PD-1 antibody every 2 weeks;

(b) 8 mg of the anti-CD137 antibody every 4 weeks and 3 mg/kg of the anti-PD-1 antibody every 2 weeks;

(c) 8 mg of the anti-CD137 antibody every 8 weeks and 3 mg/kg of the anti-PD-1 antibody every 2 weeks;

(d) 8 mg of the anti-CD137 antibody every 8 weeks and 1 mg/kg of the anti-PD-1 antibody every 2 weeks;

(e) 3 mg of the anti-CD137 antibody every 4 weeks and 1 mg/kg of the anti-PD-1 antibody every 2 weeks; and

(f) 3 mg of the anti-CD137 antibody every 8 weeks and 3 mg/kg of the anti-PD-1 antibody every 2 weeks.

Patients treated according to the methods disclosed herein preferably experience improvement in at least one sign of cancer. In one embodiment, improvement is measured by a reduction in the quantity and/or size of measurable tumor lesions. In another embodiment, lesions can be measured on chest x-rays or CT or MRI films. In another embodiment, cytology or histology can be used to evaluate responsiveness to a therapy.

In one embodiment, the patient treated exhibits a complete response (CR), a partial response (PR), stable disease (SD), immune-related complete disease (irCR), immune-related partial response (irPR), or immune-related stable disease (irSD). In another embodiment, the patient treated experiences tumor shrinkage and/or decrease in growth rate, i.e., suppression of tumor growth. In another embodiment, unwanted cell proliferation is reduced or inhibited. In yet another embodiment, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

In other embodiments, administration of effective amounts of the anti-CD137 antibody and the anti-PD-1 antibody according to any of the methods provided herein produces at least one therapeutic effect selected from the group consisting of reduction in size of a tumor, reduction in number of metastatic lesions appearing over time, complete remission, partial remission, or stable disease. In still other embodiments, the methods of treatment produce a comparable clinical benefit rate (CBR=CR+PR+SD≧6 months) better than that achieved by an anti-CD137 antibody or an anti-PD-1 antibody alone. In other embodiments, the improvement of clinical benefit rate is about 10%, 20%, 30%, 40%, 50%, 60%, 70%, 80% or more compared to an anti-CD137 antibody or an anti-PD-1 antibody alone.

### Kits and Unit Dosage Forms

Also provided herein are kits which include a pharmaceutical composition containing an anti-CD137 antibody, such as urelumab, and an anti-PD-1 antibody, such as nivolumab, and a pharmaceutically-acceptable carrier, in a therapeutically effective amount adapted for use in the preceding methods. The kits optionally can also include instructions, e.g., comprising administration schedules, to allow a practitioner (e.g., a physician, nurse, or patient) to administer the composition contained therein to administer the composition to a patient having a cancer (e.g., a solid tumor or B cell lymphoma). The kit can also include a syringe.

Optionally, the kits include multiple packages of the single-dose pharmaceutical compositions each containing an effective amount of the anti-CD137 antibody or the anti-PD-1 antibody for a single administration in accordance with the methods provided above. Instruments or devices necessary for administering the pharmaceutical composition(s) also may be included in the kits. For instance, a kit may provide one or more pre-filled syringes containing an amount of the anti-CD137 antibody or the anti-PD-1 antibody.

In one embodiment, the present invention provides a kit for treating a cancer (e.g., a solid tumor or B cell lymphoma) in a human patient, the kit comprising: (a) a dosage ranging from 0.1 to 10 mg/kg body weight of an anti-PD-1 antibody (e.g., nivolumab) or an antigen-binding portion thereof; (b) a dosage ranging from 1 to 10 mg of an anti-CD137 antibody (e.g., urelumab) or an antigen-binding portion thereof; and (c) instructions for using the anti-PD-1 antibody and the anti-CD137 antibody in a method of the present invention. In certain specific embodiments, the dosage of the anti-PD-1 antibody of the kit is 1 or 3 mg/kg body weight. In certain specific embodiments, the dosage of the anti-CD137 antibody of the kit is 3 or 8 mg.

The following examples are merely illustrative and should not be construed as limiting the scope of this disclosure in any way as many variations and equivalents will become apparent to those skilled in the art upon reading the present disclosure.

The contents of all references, GENBANK® entries, patents and published patent applications cited throughout this application are expressly incorporated herein by reference.

### Embodiments of the Present Invention

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the cancer is a solid tumor.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the solid tumor is selected from melanoma, prostate cancer, non-small cell lung cancer, colorectal cancer, head and neck squamous cell carcinoma, renal cell carcinoma, gastric carcinoma, glioblastoma, and Non-Hodgkin's Lymphoma.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the cancer is a B cell lymphoma.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the cancer is a B-cell non-Hodgkin's lymphoma.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody cross-competes with nivolumab for binding to human PD-1.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody is a chimeric, humanized or human monoclonal antibody or a portion thereof.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody comprises a heavy chain constant region which is of a human IgG1, IgG2 and IgG4 isotype, or variants thereof.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody is nivolumab.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody is administered at a dose ranging from 0.1 to 10.0 mg/kg body weight once every 2, 3 or 4 weeks.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody is administered at a dose of 1 or 3 mg/kg body weight once every 2 weeks.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-CD137 antibody cross-competes with urelumab for binding to human CD137.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-CD137 antibody is a chimeric, humanized or human monoclonal antibody or a portion thereof.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-CD137 antibody comprises a heavy chain constant region which is of a human IgG1 isotype, IgG2, IgG4 isotype, or variant thereof.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-CD137 antibody is urelumab.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-CD137 antibody is administered at a dose ranging from 1 to 10 mg once every 4 or 8 weeks.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-CD137 antibody is administered at a dose of 3 or 8 mg once every 4 weeks.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the method comprises at least one treatment cycle of eight weeks.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the method comprises at least one treatment cycle of eight weeks, and further wherein the anti-PD-1 antibody is administered on Days 1, 15, 29, and 43 of each cycle.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the method comprises at least one treatment cycle of eight weeks, and further wherein the anti-CD137 antibody is administered on Days 1 and 29 of each cycle or Day 1 of each cycle.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the method comprises at least one treatment cycle of eight weeks, and further wherein the method comprises up to 12 treatment cycles of eight weeks.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the method comprises at least one treatment cycle of eight weeks, and further wherein the anti-PD-1 antibody and the anti-CD137 antibody are administered during the first three cycles.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody and the anti-CD137 antibody are each formulated for intravenous administration.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody and the anti-CD137 antibody are administered sequentially to the subject, when both antibodies are administered on the same day.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody and the anti-CD137 antibody are administered sequentially to the subject, when both antibodies are administered on the same day, wherein the anti-PD-1 antibody and the anti-CD137 antibody are administered within 30 minutes of each other.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody and the anti-CD137 antibody are administered sequentially to the subject, when both antibodies are administered on the same day, wherein the anti-PD-1 antibody is administered prior to administration of the anti-CD137 antibody.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody and the anti-CD137 antibody are administered sequentially to the subject, when both antibodies are administered on the same day, wherein the anti-CD137 antibody is administered prior to administration of the anti-PD-1 antibody.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 and the anti-CD137 antibody are administered concurrently to the subject, when both antibodies are administered on the same day.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 and the anti-CD137 antibody are administered concurrently to the subject, when both antibodies are administered on the same day, and further wherein the anti-PD-1 antibody and the anti-CD137 antibody are administered in separate compositions.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 and the anti-CD137 antibody are administered concurrently to the subject, when both antibodies are administered on the same day, and further wherein the anti-PD-1 antibody and the anti-CD137 antibody are admixed as a single composition.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody and the anti-CD137 antibody are administered at the following dosing regimens:

(a) 3 mg of the anti-CD137 antibody every 4 weeks and 3 mg/kg of the anti-PD-1 antibody every 2 weeks;

(b) 8 mg of the anti-CD137 antibody every 4 weeks and 3 mg/kg of the anti-PD-1 antibody every 2 weeks;

(c) 8 mg of the anti-CD137 antibody every 8 weeks and 3 mg/kg of the anti-PD-1 antibody every 2 weeks;

(d) 8 mg of the anti-CD137 antibody every 8 weeks and 1 mg/kg of the anti-PD-1 antibody every 2 weeks;

(e) 3 mg of the anti-CD137 antibody every 4 weeks and 1 mg/kg of the anti-PD-1 antibody every 2 weeks; or

(f) 3 mg of the anti-CD137 antibody every 8 weeks and 3 mg/kg of the anti-PD-1 antibody every 2 weeks.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, comprising:

(a) an induction phase, wherein the anti-PD-1 and anti-CD137 antibodies are administered; followed by

(b) a maintenance phase, wherein no anti-CD137 antibody is administered and the anti-PD-1 antibody is repeatedly administered.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the method produces at least one therapeutic effect selected from a reduction in size of a tumor, reduction in number of metastatic lesions over time, complete response, partial response, and stable disease.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein administration of the anti-PD-1 antibody and the anti-CD137 antibody is continued for as long as clinical benefit is observed or until unmanageable toxicity or disease progression occurs.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-CD137 antibody comprises:

(a) a heavy chain variable region CDR1 comprising SEQ ID NO: 5;

(b) a heavy chain variable region CDR2 comprising SEQ ID NO: 6;

(c) a heavy chain variable region CDR3 comprising SEQ ID NO: 7;

(d) a light chain variable region CDR1 comprising SEQ ID NO: 8;

(e) a light chain variable region CDR2 comprising SEQ ID NO: 9; and

(f) a light chain variable region CDR3 comprising SEQ ID NO: 10.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-CD137 antibody comprises:

(a) a heavy chain variable region CDR1 comprising SEQ ID NO: 5;

(b) a heavy chain variable region CDR2 comprising SEQ ID NO: 6;

(c) a heavy chain variable region CDR3 comprising SEQ ID NO: 7;

(d) a light chain variable region CDR1 comprising SEQ ID NO: 8;

(e) a light chain variable region CDR2 comprising SEQ ID NO: 9; and

(f) a light chain variable region CDR3 comprising SEQ ID NO: 10, wherein the anti-CD137 antibody comprises heavy and light chain variable regions comprising the sequences set forth in SEQ ID NOs: 3 and 4, respectively.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-CD137 antibody comprises:

(a) a heavy chain variable region CDR1 comprising SEQ ID NO: 5;

(b) a heavy chain variable region CDR2 comprising SEQ ID NO: 6;

(c) a heavy chain variable region CDR3 comprising SEQ ID NO: 7;

(d) a light chain variable region CDR1 comprising SEQ ID NO: 8;

(e) a light chain variable region CDR2 comprising SEQ ID NO: 9; and

(f) a light chain variable region CDR3 comprising SEQ ID NO: 10, wherein the anti-CD137 antibody comprises heavy and light chains comprising the sequences set forth in SEQ ID NOs: 1 and 2, respectively.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody comprises:

(a) a heavy chain variable region CDR1 comprising SEQ ID NO: 15;

(b) a heavy chain variable region CDR2 comprising SEQ ID NO: 16;

(c) a heavy chain variable region CDR3 comprising SEQ ID NO: 17;

(d) a light chain variable region CDR1 comprising SEQ ID NO: 18;

(e) a light chain variable region CDR2 comprising SEQ ID NO: 19; and

(f) a light chain variable region CDR3 comprising SEQ ID NO: 20.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody comprises:

(a) a heavy chain variable region CDR1 comprising SEQ ID NO: 15;

(b) a heavy chain variable region CDR2 comprising SEQ ID NO: 16;

(c) a heavy chain variable region CDR3 comprising SEQ ID NO: 17;

(d) a light chain variable region CDR1 comprising SEQ ID NO: 18;

(e) a light chain variable region CDR2 comprising SEQ ID NO: 19; and

(f) a light chain variable region CDR3 comprising SEQ ID NO: 20, wherein the anti-PD-1 antibody comprises heavy and light chain variable regions comprising the sequences set forth in SEQ ID NOs: 13 and 14, respectively.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-PD-1 antibody comprises:

(a) a heavy chain variable region CDR1 comprising SEQ ID NO: 15;

(b) a heavy chain variable region CDR2 comprising SEQ ID NO: 16;

(c) a heavy chain variable region CDR3 comprising SEQ ID NO: 17;

(d) a light chain variable region CDR1 comprising SEQ ID NO: 18;

(e) a light chain variable region CDR2 comprising SEQ ID NO: 19; and

(f) a light chain variable region CDR3 comprising SEQ ID NO: 20,

wherein the anti-PD-1 antibody comprises heavy and light chains comprising the sequences set forth in SEQ ID NOs: 11 and 12, respectively.

A kit for treating a subject afflicted with a cancer, comprising:

(a) a dosage ranging from 0.1 to 10 mg/kg body weight of an anti-PD-1 antibody or an antigen-binding portion thereof;

(b) a dosage ranging from 1 to 10 mg of an anti-CD137 antibody or an antigen-binding portion thereof; and

(c) instructions for using the anti-PD-1 antibody and the anti-CD137 antibody in the method of any of embodiments referenced herein.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-CD137 antibody comprises a heavy chain constant region which is a human IgG1 isotype.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-CD137 antibody comprises a heavy chain constant region which is a human IgG2 isotype.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-CD137 antibody comprises a heavy chain constant region which is a human IgG4 isotype, or variant thereof.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-CD137 antibody binds to an CD137 epitope that promotes Fc receptor engagement and results in CD137 agonism.

A method for treating a subject afflicted with a cancer, comprising administering to the subject a combination of therapeutically effective amounts of:

(a) a monoclonal antibody or an antigen-binding portion thereof that binds specifically to a Programmed Death-1 (PD-1) receptor; and

(b) a monoclonal antibody or an antigen-binding portion thereof that binds specifically to CD137, wherein the anti-CD137 antibody binds to an CD137 epitope that promotes crosslinking and results in CD137 agonism.

### Example 1—Pre-Clinical Results Utilizing Murine Anti-PD-1 and Anti-CD137 Antibodies

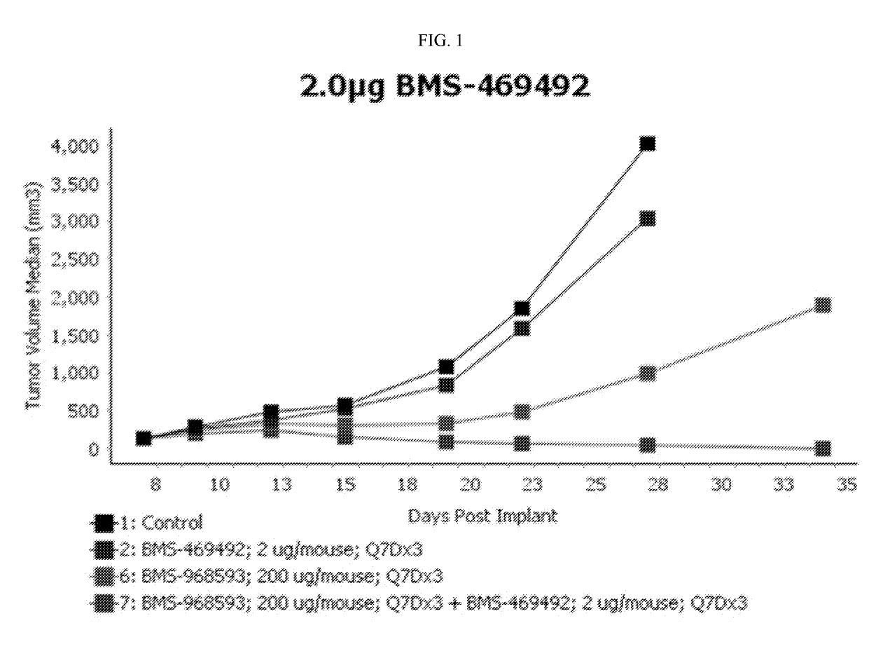

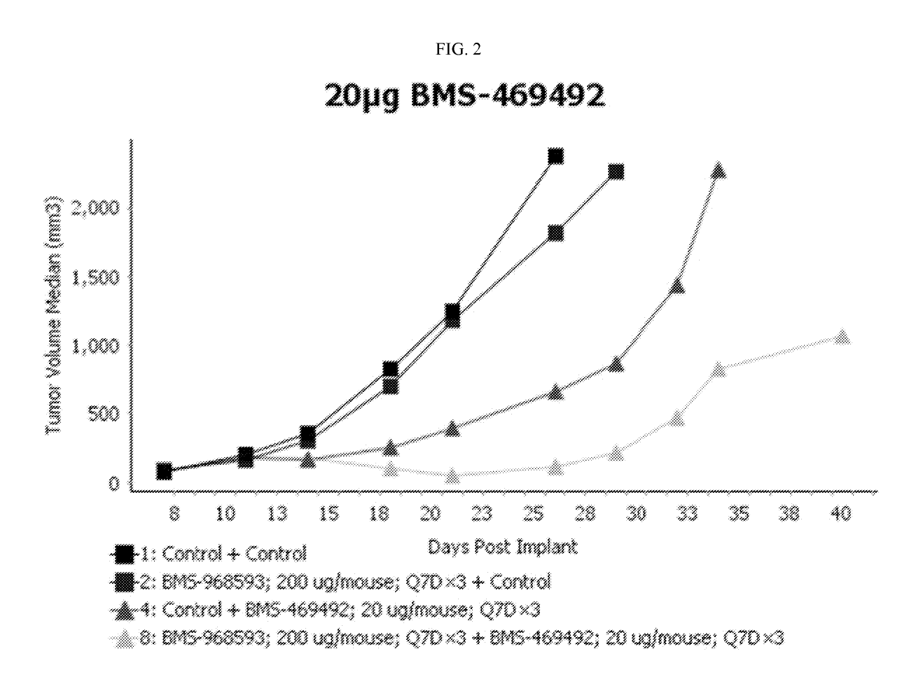

The combination of anti-CD137 and anti-PD-1 antibodies was tested for the anti-tumor efficacy in murine solid tumor models. Murine anti-PD-1 and anti-CD137 antibodies were used in the studies. The rationale is to utilize pharmaceutical manipulation to coordinately reverse T cell inhibition by blocking the PD-1 pathway using a PD-1 blocking mAb while at the same time stimulating T cell proliferation using an agonistic anti-CD137 mAb. Multiple tumor models have been tested using the mouse anti-PD-1 (BMS-968593) and mouse anti-CD137 (BMS-469692) including M109 lung adenocarcinoma, MC38 colon cancer, CT26 colon cancer, and SA1N fibrosarcoma models. In all of the models, the mouse anti-PD-1 showed modest activity as monotherapy, but the combination of mouse anti-CD137 and mouse anti-PD-1 showed synergy in terms of greater efficacy than anti-PD1 alone. In an ID8 mouse ovarian cancer model, combination of anti-CD137/PD-1 mAb was superior to single mAb administration resulting in a doubling of overall survival mice with tumors (Wei et al., PLoS ONE, 8(12):e84927 (2013)). The MC38 and M109 models are displayed as FIG. 1 and FIG. 2 respectively.

The understanding of the mechanism of the observed synergy of anti-CD137 antibody and anti-PD1 antibody is still evolving. An increased presence of tumor-infiltrating lymphocytes (TILs) is thought to correlate with improved responses in cancer, supporting the notion that spontaneous T cells with antitumor activity can accumulate within neoplastic lesions and control tumor growth. In an ID8 mouse ovarian cancer model, mice treated with this mAb combination have a significantly increased frequency and total number of CD8+ T cells both in the peritoneal lavage and spleens, and these cells are functional as demonstrated by antigen-specific cytolytic activity and IFN-γ production (Wei et al., supra).

These preliminary data suggest that this combination leads to increased frequency of functional, antigen directed T cells trafficking to the tumor microenvironment, and along with reversal of T cell inhibition, could result in greater anti-tumor synergy.

In non-clinical testing, the combination of urelumab and nivolumab does not result in a synergistic adverse event profile.

These results provide pre-clinical data to support the potential benefit of combining anti-CD137 and anti-PD-1 antibodies in a clinical trial.

### Example 2—Preclinical Pharmacology and Toxicity of Urelumab and Nivolumab

### Pharmacology of Urelumab

Urelumab ((BMS-663513) is a fully human agonist IgG4K isotype monoclonal antibody specific to the human CD137 receptor. Urelumab has a molecular weight of 150 kDa. Antibodies to the human CD137 receptor do not cross-react with the murine receptor and vice-versa. Therefore, an anti-murine CD137 receptor antibody homolog, BMS-469492, was used for evaluation in murine tumor models. Both urelumab and BMS-469492 (mouse anti-CD137) are agonistic antibodies which do not block the interaction of CD137 with its ligand, CD137L. Both the human and the mouse anti-CD137 antibodies increase IFNγ secretion from T cells activated with anti-CD3 in an in vitro functional assay.

The functional effects of urelumab on human and monkey T cells and peripheral blood mononuclear cells (PBMC) were determined by measuring IFNγ production by human T cells or monkey PBMC from healthy donors that were stimulated with anti-CD3 antibody (0.5-1 μg/mL)±anti-human CD137 antibodies. Urelumab demonstrated co-stimulatory properties yielding higher levels of IFNγ in human and monkey cells compared to controls (anti-CD3+/control IgG).

Endogenous CD137 provides a co-stimulatory signal to T cells which results in enhancement of T-cell survival, T-cell proliferation and cytokine synthesis. To determine whether urelumab agonist antibody could elicit these same biologic effects, human T cells stimulated with anti-CD3±urelumab at concentrations known to induce IFNγ synthesis were stained with annexin-V and propidium iodide to determine the number of live cells (annexin-V/propidium iodide negative) and with cyclin D2 to assay the cell cycle status of treated cells. Concentrations of urelumab ranging from 0.4-10 μg/mL resulted in an increase in the number of live cells by approximately 1.8 to 2-fold, and yielded a significant increase in the number of cyclin D2 expressing T cells by 2.5 to 3-fold), confirming the costimulatory effect of urelumab.

### Pharmacology of Nivolumab

Nivolumab (BMS-936558) is a fully human, IgG4 (kappa) isotype monoclonal antibody that binds to PD-1 with nanomolar affinity (KD=3.06 nM) and a high degree of specificity, thus precluding binding to its ligands PD-L1 and PD-L2. Nivolumab does not bind other related family members, such as BTLA, CTLA-4, ICOS or CD28. Pre-clinical testing of nivolumab demonstrated that binding to PD-1 results in enhanced T cell proliferation and release of interferon-gamma (IFN-gamma) in vitro.

### Toxicity of Urelumab

Intravenous administration of urelumab to monkeys at single doses up to 100 mg/kg (AUC(0-INF) 534,000 μg h/mL) or repeated doses up to 100 mg/kg (AUC≦833,000 μg h/mL), every two weeks for up to 9 month, did not result in drug-related toxicity. In mice, the liver was identified as the principal target organ with the murine homolog BMS-469492. The liver lesion occurred after single exposures to BMS-469492 at doses≧0.2 mg/kg and was characterized as minimal to moderate subacute inflammation of the liver and hepatocellular necrosis. Alanine or aspartate aminotransferases (ALT or AST) were generally sensitive and specific markers of this injury. Based upon the results from investigative studies the liver lesion was consistent with local IFN-γ-mediated injury and likely represents a mechanism-related effect of CD137 modulation in mice. The NOEL for liver inflammation in mice was achieved following a single dose of BMS-469492 at 0.1 mg/kg. No therapeutic window for this toxicity exists in the mouse efficacy models. No liver toxicity was observed in the monkey studies with urelumab.

Multiple-dose administration of the murine homolog BMS-469492 in mice induced liver and skin toxicities. There was minimal to moderate chronic inflammation and hepatocellular single-cell necrosis, increased ALT and AST, and pigment within Kupffer cells. The severity of the reversible hepatotoxicity in mice did not progress with increasing dose level, increasing number of doses, or with different dose frequencies. The NOEL for liver inflammation with multiple-dose administration of BMS-469492 to mice was not determined. The hepatic toxicity was not exacerbated by co-administration of known inflammatory agents, such as lipopolysaccharide (LPS) or acetaminophen (APAP). Instead, pre-treatment with BMS-469492 showed a trend to prevent or dampen the acute centrilobular hepatocellular necrosis induced by APAP.

### Toxicity of Nivolumab

Toxicology studies in cynomolgus monkeys revealed that nivolumab was well tolerated at doses up to 50 mg/kg given twice weekly for 27 doses. Drug related findings were limited to a reversible decrease in triiodothyronine (T3) by 28%, without concomitant abnormalities in other markers of thyroid function.

Preliminary new non-clinical safety findings of adverse pregnancy outcomes and infant losses in the absence of overt maternal toxicity have been reported. The findings of increased late stage pregnancy loss and early infant deaths/euthanasia in nivolumab exposed pregnant monkeys suggest a potential risk to human pregnancy if there is continued treatment with nivolumab during pregnancy.

### Example 3—Clinical Pharmacology and Safety of Urelumab and Nivolumab

### Urelumab

Four studies in humans have been conducted using urelumab: 2 monotherapy studies (a Phase 1 study, CA186001, in subjects with solid malignancies and a Phase 2 study, CA186006, in subjects with advanced melanoma) and 2 combination therapy phase 1 studies [CA186004 (combining urelumab with carboplatin and paclitaxel in subjects with solid malignancies) and CA186005 (combining urelumab with radiation and carboplatin with paclitaxel in subjects with non small cell lung cancer)].

Among the subjects treated with urelumab in monotherapy studies, drug-related AEs were reported in about 79.1% subjects. Drug-related≧Grade 3 AEs were reported in about 27.5% subjects. The most frequently reported drug-related AEs were fatigue, increased AST, increased ALT, rash nausea, pruritus, pyrexia, decreased appetite, and diarrhea. Other drug-related AEs included headache, decreased platelets, asthenia, neutropenia, febrile neutropenia, and thrombocytopenia.

A maximum tolerated dose of urelumab administered intravenously on an every 3 week schedule was not formally defined during dose escalation studies. Drug-related hepatotoxicity was reported previously during the trials. Subsequent studies showed the significantly improved hepatic safety profile at doses below 1 mg/kg versus that of doses at or above 1 mg/kg.

Evaluation of the safety data revealed that drug-related hepatotoxicity is the most frequent clinically significant drug-related AE experienced among subjects treated with urelumab and is dose dependent. Exposure response analysis revealed that the occurrence and severity of hepatoxicity may be correlated with exposure (Cavg) and is substantially increased at doses≧1 mg/kg. Doses of urelumab<1 mg/kg every 3 weeks resulted in a low frequency of ≧Grade 3 hepatotoxicity, whereas an urelumab dose of ≧1 mg/kg every 3 weeks resulted in more frequent ≧Grade 3 hepatotoxicity

### Nivolumab

The overall safety experience with nivolumab, as monotherapy or in combination with other therapeutics, is based on experience in approximately 1,500 subjects treated to date. For monotherapy, the safety profile is similar across tumor types. The one exception is pulmonary inflammation AEs which may be numerically greater in subjects with NSCLC because in some cases it can be difficult to distinguish between nivolumab related and unrelated causes of pulmonary symptoms and radiographic changes. There was no pattern in the incidence, severity, or causality of AEs to nivolumab dose level.

In several ongoing clinical trials, the safety of nivolumab in combination with other therapeutics such as ipilimumab, cytotoxic chemotherapy, anti-angiogenics and targeted therapies is being explored. Most studies are ongoing and as such, the safety profile of nivolumab combinations continues to evolve. The most advanced combination under development is nivolumab and ipilimumab in subjects with MEL. Thus far, the combination of both agents results in a safety profile with similar types of AEs as either agent alone, but in some cases with greater frequency.

Overall, the safety profile of nivolumab monotherapy as well as combination therapy is manageable and generally consistent across completed and ongoing clinical trials with no MTD reached at any dose tested, up to 10 mg/kg. There was no pattern in the incidence, severity, or causality of AEs to nivolumab dose level. Most AEs were low grade (grade 1 to grade 2) with relatively few related high grade (grade 3 to grade 4) AEs. Most high grade events were manageable with the use of corticosteroids or hormone replacement therapy for endocrinopathies. Nivolumab should not be used in subjects with active autoimmune disease given the mechanism of action of the antibody.

Nivolumab, alone or in combination with another cancer therapy, has demonstrated clinical activity in response evaluable subjects with a variety of solid tumor malignancies, such as prostate cancer, MEL, NSCLC, renal cell carcinoma, SCCHN, HCC, CRC, GBM, and NHL.

### Example 4—Clinical Pharmacokinetics of Urelumab and Nivolumab

### Urelumab

The CA186001 first-in-human study showed that over the dose range studied (0.3 mg/kg to 15 mg/kg), urelumab concentrations were quantifiable within approximately 0.5 hour and peak concentrations occurred between 1 and 5 hours. The CA186001 and CA186011 studies showed that at 0.1 and 0.3 mg/kg (the dose range to be tested in this study), the mean serum elimination half-life of urelumab in subjects with solid malignancies was approximately 125-135 hours (5.2-5.6 days). Serum urelumab Cₘₐₓ and AUC increased in proportion to dose when administered at 0.1 to 0.3 mg/kg.

### Nivolumab

A single dose pharmacokinetic analysis of 39 subjects with cancer given nivolumab at 0.3, 1, 3 and 10 mg/kg revealed that the median Tₘₐₓ across single doses ranged from 1.6 to 3 hours with individual values ranging from 0.9 to 7 hours. The pharmacokinetics of nivolumab were linear in the range of 0.3 to 10 mg/kg with dose proportional increases in maximum serum concentration (Cₘₐₓ) and area under the concentration-time curve from time zero to infinity (AUCINF), with low to moderate inter-subject variability observed at each dose level. The mean terminal elimination half-life of nivolumab was 17 to 25 days, which is consistent with the half-life of endogenous IgG4. Both the elimination and distribution of nivolumab were independent of the dose.

### Example 5—A Phase 1/2 Dose Escalation and Cohort Expansion Study of the Safety and Tolerability of Urelumab Administered in Combination with Nivolumab in Cancer Patients

### Objectives

The primary objective of the study is to assess the safety and tolerability of urelumab given in combination with nivolumab and to identify dose limiting toxicities (DLTs) and the maximally tolerated dose (MTD) of the combination, in subjects with advanced (metastatic and/or unresectable) solid tumors and B cell lymphomas.

Secondary objectives include assessing the preliminary anti-tumor activity of the combination of urelumab and nivolumab in subjects with advanced solid tumors and B cell lymphomas, characterizing the pharmacokinetics (PK) of urelumab and nivolumab when co-administered, monitoring immunogenicity of urelumab and nivolumab administered as combination therapy.

Additional exploratory objectives include assessing the pharmacodynamic effects of urelumab as a function of exposure when given in combination with nivolumab in peripheral blood and tumor tissue, exploring potential associations between biomarker measures and anti-tumor activity, assessing the overall survival (OS) following the start of therapy with the combination of urelumab and nivolumab.

### Study Design and Duration

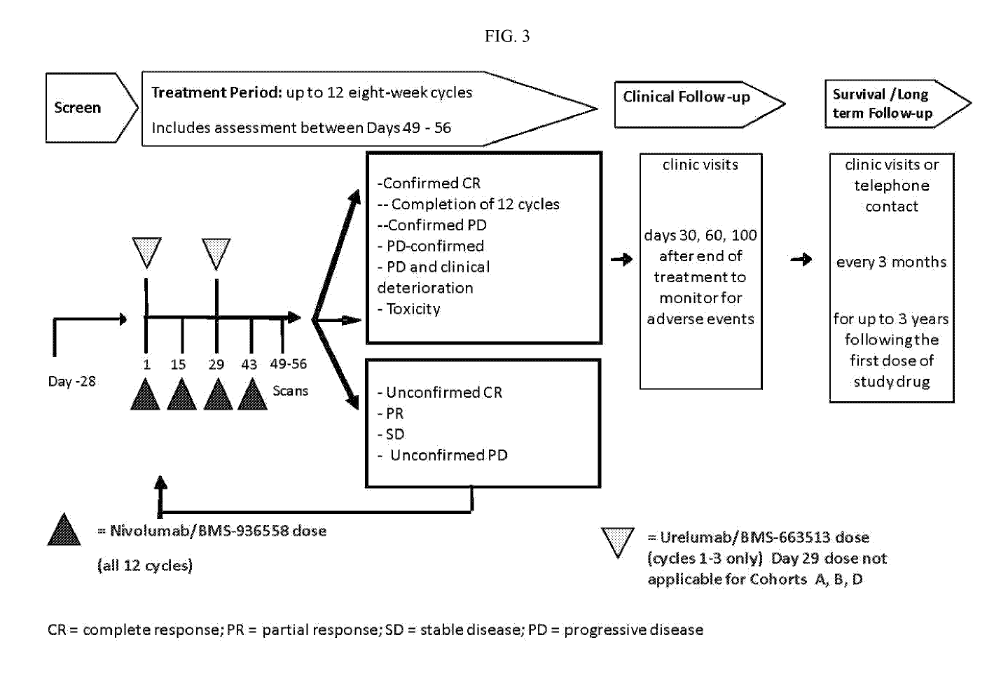

The design is for a phase 1/2 open label study. The first phase of the study consists of a dose escalation assessment of the safety and tolerability of urelumab administered with nivolumab in subjects with advanced solid tumors or B-cell NHL. The second phase of the study includes a 2-stage cohort expansion in 4 tumor types: melanoma (MEL), non-small cell lung cancer (NSCLC), head and neck squamous cell carcinoma (SCCHN), and diffuse large B cell lymphoma (DLBCL). Expansion cohorts are explored at the maximally tolerated dose (MTD), highest administered dose (HAD), or at an alternative dose/regimen as determined by the investigators and the sponsor. The study design schema is below in FIG. 3.

Subjects complete up to four periods of the study as follows: (1) Screening (up to 28 days); (2) Treatment (up to a maximum of 12 cycles of study therapy); (3) Clinical Follow-up (100 days following study drug discontinuation); and (4) Survival/Long-term Follow-up (up to 3 years following the first dose of study drug). The total time on study for any individual subject is expected to be approximately 3.1 years.

The Treatment Period consists of up to 12 eight-week treatment cycles (96 weeks). Nivolumab is given every 2 weeks up to all 96 weeks. Urelumab is given every 4 or every 8 weeks up to 24 weeks. The first 3 treatment cycles are comprised of 4 doses of nivolumab per cycle and either 1 or 2 doses of urelumab per cycle. Nivolumab is administered on Days 1, 15, 29, and 43; and urelumab is administered on either Day 1 or on Days 1 and 29 of each treatment cycle depending on the treatment cohort. On days where both study drugs are given, nivolumab is given first followed by urelumab within 30 minutes of completing the infusion of nivolumab. Treatment Cycles 4-12 are comprised of 4 doses per cycle of nivolumab as monotherapy.

Following each treatment cycle, the decision to treat a subject with additional cycles of study therapy is based on tumor assessment (evaluation performed between Days 49 and 56 of each cycle and completed before the first dose in the next cycle). Tumor progression or response endpoints are assessed using RECIST 1.1. Treatment beyond initial investigator-assessed progression (either clinical or radiographical) is not permitted in Lymphoma patients. Treatment beyond initial investigator-assessed progression (either clinical or radiographical) is permitted only in subjects with solid tumors if the subject has an investigator-assessed clinical benefit and is tolerating study drug. Subjects with a response of unconfirmed progressive disease (PD), stable disease (SD), partial response (PR), or complete response (CR) at the end of a given cycle continue to the next treatment cycle. Subjects generally are allowed to continue study therapy until the first occurrence of either: 1) completion of the maximum number of cycles, 2) confirmed PD, 3) clinical deterioration suggesting that no further benefit from treatment is likely, 4) intolerability to therapy; or 5) the subject meets criteria for discontinuation.

Subjects that discontinue the treatment phase enter the Clinical Follow-up period. Subjects must be followed for at least 100 days after the last dose of therapy.

After completion of the Clinical Follow-up period, subjects then enter the Survival/Long-Term Follow-up period. During this period, clinic visits or telephone contact every 3 months are performed to assess survival status. The duration of survival follow-up is 3 years following the first dose of the study drug. In addition, subjects who discontinue study drug for reasons other than progression will continue to have tumor assessments completed every 12 weeks for the first year and then continue to receive scans per standard of care guidelines for follow-up or at a minimum of every 6 months until disease progression or withdrawal of consent. Data from imaging assessments for subjects who have ongoing clinical benefit may continue to be collected after subjects discontinue the actual survival phase of the study. Subjects in the Survival/Long-Term Follow-up period who have progression of disease are allowed to receive tumor directed therapy as required.

### Dose Escalation

A 3+3+3 design is used to assess the safety of urelumab given in combination with nivolumab. The cohorts for dose escalation are provided in Table 1. Potential alternate cohorts are provided in Table 3. The Dose Limiting Toxicity (DLT) observation period lasts for 8 weeks. The DLT evaluation period is defined as up to 8 weeks after administration of the first combination dose of nivolumab and urelumab, and includes administration of at least one dose of nivolumab monotherapy during this interval. This interval is based upon inclusion of the earliest times to onset of clinically significant adverse events attributed to study drug, and also allows for a substantial amount of time for unexpected toxicities related to dosing regimen to emerge.

Approximately three subjects are treated initially at each dose regimen. In order to assure sufficient evaluable subjects per cohort an additional subject may be added to a cohort (i.e., enroll a fourth subject in a cohort of 3).

Cohort tolerability assessment and subsequent dose escalation, if indicated, occur when the minimum number of subjects required to evaluate tolerability have completed the 8 week DLT period. However, if any additional subject experiences an event that would, per protocol, result in either cohort expansion or the halting of dose escalation, the escalation rules as defined below in Table 2 is followed.

TABLE 1
Doses/Regimens During Dose Escalation

| Cohort | | | |
|---|---|---|---|
| Number | Total Subjects^{a} | urelumab | nivolumab |
| 1 | n = approximately 3-9 | 3 mg IV every | 3 mg/kg IV every 2 |
| | | 4 weeks | weeks |
| 2 | n = approximately 3-9 | 8 mg IV every | 3 mg/kg IV every 2 |
| | | 4 weeks | weeks |
| Total | n = approximately 6-18 | | |

^{a}3-9 subjects will be enrolled during dose escalation. Additional subjects may be added to each dose level after completion of the dose escalation period of the study for a total of up to 12 subjects per dose level

Table 2 outlines the decision rules for dose escalation based on the number of subjects and observed DLTs. No intra-subject dose escalation or reduction is allowed. Subjects who withdraw from the study during the DLT period for reasons other than a DLT may be replaced within the same dose level/regimen. Dose escalation is based on the number of dose limiting toxicities (DLTs) experienced during the DLT observation period. If dose escalation is terminated, then the dose below that which invoked the stopping rule is declared the MTD.

TABLE 2
Decision Rules During Dose Escalation

| | Number of Evaluable Subjects/Cohort | | | | | | | |
|---|---|---|---|---|---|---|---|---|
| | 3-4 | | | 6-8 | | | 9-12 | |
| Total Number | 0 | 1 | 2 or more | 1 | 2 | 3 or more | 2 | 3 or more |
| of Observed | | | | | | | | |
| DLTs | | | | | | | | |
| Decision Rule | Dose Escalate | Enroll | Dose exceeds | Dose Escalate | Enroll | Dose exceeds | Dose Escalate | Dose exceeds |
| | | additional | MTD | | additional | MTD | | MTD |
| | | subjects at in | | | subjects at in | | | |
| | | cohort to reach | | | cohort to reach | | | |
| | | at least 6 | | | at least 9 | | | |
| | | subjects | | | subjects | | | |

If either Cohort 1 or Cohort 2 exceeds the MTD, alternate treatment regimens may be explored during dose escalation or cohort expansion (listed in Table 3).

TABLE 3
Alternate Treatment Regimens

| Cohort | urelumab | nivolumab |
|---|---|---|
| A | 8 mg IV every 8 weeks | 3 mg/kg IV every 2 weeks |
| B | 8 mg IV every 8 weeks | 1 mg/kg IV every 2 weeks |
| C | 3 mg IV every 4 weeks | 1 mg/kg IV every 2 weeks |
| C | 3 mg IV every 8 weeks | 3 mg/kg IV every 2 weeks |

All available clinical and laboratory data observed during dose escalation are reviewed to determine the alternative treatment regimen listed in Table 3 to be evaluated. The nature, time of onset, and time to resolution of DLTs observed are reviewed in the context of the current safety data from the respective urelumab and nivolumab trials. After review of this data, and after consultation between the investigators and the sponsor, the identified alternative treatment regimens may be evaluated.

### Cohort Expansion

The purpose of the cohort expansions is to gather additional safety, tolerability, preliminary efficacy and pharmacodynamic information regarding the combination of urelumab and nivolumab. Once the MTD of combined administration of urelumab and nivolumab has been defined, cohort expansions is initiated.

Expansion cohorts follow a two-stage design as defined below in Table 4.

TABLE 4
Cohort Expansion

| Tumor Types | Stage 1 Expansion | | Stage 2 Expansion |
|---|---|---|---|
| Non-small | 1:1 | →20 subjects at | Up to approximately |
| Cell Lung | Randomization | MTD | 20 subjects at the |
| Cancer | | →20 subjects at | preferred cohort |
| (NSCLC) | | alternate schedule | from Stage 1 |
| Melanoma | 1:1 | →20 subjects at | Up to approximately |
| (MEL) | Randomization | MTD | 20 subjects at the |
| | | →20 subjects at | preferred cohort |
| | | alternate schedule | from Stage 1 |
| Head and Neck | 1:1 | →20 subjects at | Up to approximately |
| Squamous Cell | Randomization | MTD | 20 subjects at the |
| Carcinoma | | →20 subjects at | preferred cohort |
| (SCCHN) | | alternate schedule | from Stage 1 |
| Diffuse Large | 1:1 | →20 subjects at | Up to approximately |
| B Cell | Randomization | MTD | 20 subjects at the |
| Lymphoma | | →20 subjects at | preferred cohort |
| (DLBCL) | | alternate schedule | from Stage 1 |

Stage 1 of the expansion phase includes a 1:1 randomization between 2 cohorts (20 subjects each) followed by Stage 2: continued enrollment of up to approximately 20 subjects in one of the two randomized cohorts. The randomization includes the MTD or HAD cohort from dose escalation and an alternative regimen (Table 3). The alternative treatment regimen included during Stage 1 is selected using available urelumab data including data collected during the dose escalation phase of the trial. Should no MTD be reached during dose escalation, the default cohorts to be evaluated during Stage 1 are the MTD/HAD and Cohort A.

Randomized cohort expansions are included to assess the potential impact of different treatment and dosing regimens of the combination of urelumab and nivolumab on benefit or risk.

The safety, tolerability, and preliminary efficacy data from Stage 1 of the cohort expansion are evaluated per tumor type to determine the treatment regimen to be evaluated during Stage 2 (see the criteria below).

Efficacy Criteria for Moving to Stage 2

A minimum of 4 out of 20 subjects should demonstrate an objective response to study therapy in a given treatment regimen for that regimen to be considered of clinical interest. In general, if 0 to 3 responses are observed during Stage 1, that treatment regimen is discontinued and is not eligible for continued enrollment during for Stage 2 of the expansion phase for that tumor type. If both treatment regimens being explored in a given tumor type during Stage 1 have <4 responses, Stage 2 is enrolled for that tumor type.

Safety Criteria for Moving to Stage 2

If the rate of DLTs exceeds 33% in a given tumor type and given treatment regimen; the findings are discussed and further enrollment may be interrupted for that specific treatment regimen and tumor type.

If both treatment regimens in a specific tumor type meet the minimum safety and efficacy criteria for further testing, then the safety and efficacy data from the subjects enrolled in Stage 1 of the expansion phase are assessed and reviewed with participating investigators and a decision is made to select the treatment regimen to continue for Stage 2.

Following selection of the cohort to evaluate during Stage 2, up to approximately 20 additional subjects are enrolled in the regimen. The evaluation of data from Stage 1 and the decision to begin Stage 2 of the expansion phase of the study can be made up to one year following completion of the enrollment of Stage 1 for each individual tumor type.

### Dose Limiting Toxicity

The incidences of DLTs which occur within 8 weeks following the start of study therapy guide dose escalation decisions. Adverse events are graded according to the National Cancer Institute (NCI) Common Terminology Criteria for Adverse Events version 4.0 (CTCAEv4). For the purposes of subject management, drug-related adverse events (AEs) occurring at any time which meet the DLT definition lead to dose interruption and or permanent discontinuation of study drug.

### Duration of Study

The total time on study for any individual subject is expected to be approximately 3.1 years. The total duration of the study is expected to be 4.5 years from the time of the first visit of the first subject to the required survival follow-up of the last subject enrolled.

### Number of Subjects

Approximately 260 subjects are dosed.

### Study Population

Subjects at least 18 years old, who have histologic confirmation of a solid malignancy or B-cell NHL that is advanced (metastatic and/or unresectable), with measurable disease, are eligible to participate in the study. For Dose Escalation, subjects with any solid tumor type (with the exception of primary central nervous system tumors) and B-cell NHL are eligible to enroll. For Cohort Expansion, subjects must have one of the following tumor types to be eligible: NSCLC; MEL, SCCHN, or DLBCL.

### Study Assessments

Safety Outcome Measures:

Safety assessments are based on medical review of adverse event reports and the results of vital sign measurements, ECGs, physical examinations, and clinical laboratory tests. The incidence of observed adverse events is tabulated and reviewed for potential significance and clinical importance. Adverse events are assessed continuously during the study and for 100 days after the last treatment. Both AEs and laboratory tests are graded using the NCI CTCAEv4.

Efficacy Measures:

In solid tumor patients, disease assessment with computed tomography (CT) and/or magnetic resonance imaging (MRI), as appropriate, is performed at baseline and every 8 weeks until confirmed disease progression, at the completion of follow-up, or until subjects withdraw from the study. Tumor responses are derived for appropriate populations of subjects as defined by RECIST v1.1 based on recorded tumor measurements. For lymphomas, the primary efficacy assessment is objective response rate (ORR), defined as a subject achieving either a partial remission (PR) or complete remission (CR) according to the revised International Working Group Criteria for non-Hodgkin Lymphoma Cheson et al., (“Revised Response Criteria for Malignant Lymphoma”, J. Clin. Oncol., 25(5):579-586, Epub. Jan. 22, 2007). The primary efficacy assessment, along with the secondary endpoints of duration of OR, complete remission rate (CR), and progression free survival (PFS) is performed based on investigators assessments. Subjects are assessed for response by imaging (spiral CT/MRI) beginning at week 8 until disease progression. A PET scan is required to confirm CR. At the sponsor's discretion, scans and measurements may be collected and reviewed by independent radiologists at a later date, or at any time during the study.

Pharmacokinetic Measures:

The nivolumab pharmacokinetic concentrations are measured to derive the trough (Cₘᵢₙ) and end of infusion concentration at specified visits. Pharmacokinetic serum concentrations of urelumab are measured at specified time points to derive PK parameters (Cₘₐₓ, Cₘᵢₙ, Tₘₐₓ, AUC(INF), AUC(TAU), T-HALF, CL, Vss, and AI).

Immunogenicity Measures:

Serum samples to evaluate development of positive anti-drug antibody (ADA) response to urelumab and nivolumab are collected at specified time points.

Biomarker Measures:

The sample collection and biomarker assessment strategy is designed to investigate the actions of urelumab and nivolumab and the simultaneous modulation of the innate and adaptive immune systems. There are three types of specimens obtained for biomarker testing: Tumor Tissue, Whole Blood, and Serum.

(1) Tissue Biopsies:

Correlation between baseline biomarkers, e.g., PD-L1 and CD137 pos-TILs and response rate and overall survival supports rationale for combination of these agents. Tracking changes in biomarkers measured in tumor tissue during treatment is instrumental to determining the mechanisms of action of cancer therapeutics. Key analytes include immunomodulatory proteins expressed on tumor (e.g., PD-L1) and markers associated with TILs (e.g., CD3, CD8, CD137).

(2) Whole Blood for Nucleic Acids:

Whole blood is collected from all subjects on the day of first treatment to generate genomic DNA for SNP genotyping. Separate whole blood samples (PAXGENE®) are obtained at baseline and at multiple times during treatment to monitor pharmacodynamic changes in expression of immunoregulatory genes including immunoglobulins, interferon-inducible genes, and genes associated with major immune cell subtypes.

(3) Whole Blood—for PBMC-Based Flow Cytometry:

Flow cytometry is used to assess baseline and serial on treatment alterations in composition/activation status of lymphocyte subsets present in peripheral blood mononuclear cell preparations (PBMCs).

(4) Serum:

To understand the prevalence of circulating proteins and the impact they may have on the clinical activity and/or safety of nivolumab-urelumab treatment, the protein concentrations of a panel of cytokines, chemokines, and other relevant immunomodulatory, serum-soluble factors are investigated by ELISA and/or other relevant multiplex-based protein assay methods.

### Statistical Considerations

Dose Escalation:

As this is a Phase 1/2 dose escalation trial, the sample size at each dose cannot be determined exactly, as it depends on the number of observed toxicities. Approximately 3 to 9 subjects are expected to be treated during dose escalation in each cohort, and up to 12 subjects may be dosed at selected cohorts

Cohort Expansion:

During Stage 1 of cohort expansion approximately 40 subjects in each tumor type are randomized in a 1:1 ratio to receive either the MTD/HAD dose level and schedule, or an alternate schedule (20 subjects in each group). With a sample size of approximately 20 subjects in each treatment regimen, it is intended to provide a general picture of the safety of each regimen. For example, if a low grade adverse event were observed in 3 or fewer patients, the 90% 1-sided upper confidence interval would be 30%. During Stage 2 of cohort expansion, up to approximately 20 additional subjects are treated in the tumor types using the cohort selected during Stage 1. This allows for further establishment of the safety profile of the combination and a preliminary assessment of efficacy. A total of 40 subjects at the selected treatment regimen (20 from Stage 1 and 20 from Stage 2) is based on achieving a higher precision. If in a cohort of 40 subjects 12, 15, or 18 responses are observed, then the lower limit of the one-sided 90% CI for the ORR is 20%, 27%, and 34% respectively. These calculations are based on the Clopper-Pearson method for exact confidence intervals. If the true ORR in a tumor type is 50%, then with 40 subjects in a cohort there is 96% chance of observing at least 15 responses, and 92% chance of observing at least 16 responses, and there is 8% chance of observing 15 or fewer responses (false negative rate).

### Endpoints

The primary endpoint of this phase 1/2 study is safety as measured by the rate of adverse events (AEs) and Serious Adverse Events (SAEs). All subjects who receive at least one (full or partial) dose of urelumab or nivolumab are evaluated for safety during treatment and for up to 100 days in follow-up.

Secondary endpoints include efficacy, pharmacokinetics, and immunogenicity.

(1) Efficacy:

Objective response rate (ORR), and progression free survival (PFS) are assessed based on RECIST v 1.1 in subjects with solid tumors, and based on investigator assessment per revised IWG/Cheson's criteria in subjects with hematologic malignancies.

(2) Pharmacokinetics:

Urelumab maximum concentration Cₘₐₓ (μg/mL), time to maximum concentration Tₘₐₓ (hr), Area under the curve AUCTAU (μg·hr/mL), Area under the curve AUCinf (μg·hr/mL), Clearance (L/day), Volume of distribution (Vss), half-life (t½), and trough concentration Cₘᵢₙ (μg/mL) are evaluated using non-compartmental analysis in all study subjects.

(3) Immunogenicity:

Occurrence of specific ADA to urelumab and nivolumab is determined.

Exploratory endpoints include biomarkers. Biomarkers monitored in peripheral blood include multiple measures such as, but not limited to, (i) Flow cytometry of PBMCs to phenotype NK- and T-Cells; (ii) whole blood gene expression of immunoregulatory factors (e.g., IFNγ-induced genes); (iii) Genotyping (SNPs); and (iv) serum concentrations of a panel of soluble factors, including cytokines and chemokines. Biomarkers monitored using tissue include: (i) IHC for markers of tumor infiltrating lymphocytes (TILs) such as but not limited to CD137, CD4, CD8, FoxP3, CD16, and CD56 proteins; (ii) IHC for immunomodulating proteins expressed on tumor (e.g., PD-L1); (iii) IHC for proteins associated with ErbB family signaling pathways; (iv) gene expression profiling with a focus on genes associated with IFN′ signaling; and (v) Mutation status of genes which may impact response (e.g., HRAS; PIK3CA genes, EGFR genes).

### Statistical Analysis

Safety:

All recorded adverse events are listed and tabulated by system organ class, preferred term, and dose. The most current version of MedDRA is used for coding. Vital signs and clinical laboratory test results are listed and summarized by treatment. Any significant physical examination findings and results of clinical laboratory tests are listed. ECG results are evaluated by the investigator; clinically significant abnormalities are listed.

Pharmacokinetics:

Summary statistics are tabulated for the pharmacokinetic parameters of urelumab by dose/schedule and study day/week. Nivolumab end of infusion and trough (Cₘᵢₙ) concentration are tabulated by summary statistics. This data may also be pooled with other datasets for population PK analysis which are part of a separate report.

Immunogenicity Analyses:

A listing will be provided of all available immunogenicity data. Additionally, a listing of immunogenicity data from those subjects with at least one positive ADA at any time point is provided. The frequency of subjects with at least one positive ADA assessment, and frequency of subjects who develop ADA after a negative baseline assessment are provided. To examine the potential relationship between immunogenicity and safety, the frequency and type of AEs of special interest may be examined by overall immunogenicity status.

Efficacy Analyses:

Individual BOR, duration of response and PFS are listed using RECIST v1.1 criteria in solid tumors and investigator assessed revised IWG/Cheson's criteria in lymphomas. BOR outcomes are tabulated by disease type and dose. The ORR and PFS rate (e.g., at 24 weeks) and the corresponding confidence interval are provided by tumor type and treatment. The duration of response and PFS are estimated by Kaplan-Meier methodology by tumor type, depending on data availability. PFS rates at 24 weeks are similarly estimated, based on K-M methodology. Presentations of efficacy include subjects in cohort expansion and subjects in dose escalation matching those in cohort expansion by tumor type and treatment. Individual changes in the tumor burden over time are presented graphically within a tumor type. Landmark overall survival is also assessed as part of exploratory efficacy analysis, by Kaplan-Meier plots and medians for each tumor type.

Biomarker Analyses:

Exploratory Biomarker Analyses can be found under “Exploratory Endpoints”.

Pharmacodynamics:

Summary statistics for immunoregulatory activities (pharmacodynamic markers) measured in peripheral blood and tissue are tabulated by planned time point and disease type (when applicable based on data availability). Possible associations between pharmacodynamic biomarker measures of interest with i) exposure, ii) safety data (adverse events), and/or iii) anti-tumor activity of urelumab-nivolumab treatment is explored graphically and further by a linear/nonlinear mixed model, if data warrant.

Candidate Predictive Biomarkers:

The potential associations of candidate pre-treatment biomarkers measured in peripheral blood and tissue with the anti-tumor activity of urelumab treatment and/or adverse events are explored based on data availability. Methods such as, but not limited to, summaries, graphics and logistic regression may be used to further investigate such associations.

### Example 6—Pre-Clinical Results Evaluating Mono- and Combination Therapy with Murine Anti-PD-1 and Murine Anti-CD137 Antibodies Containing Different Immunoglobulin Isotypes

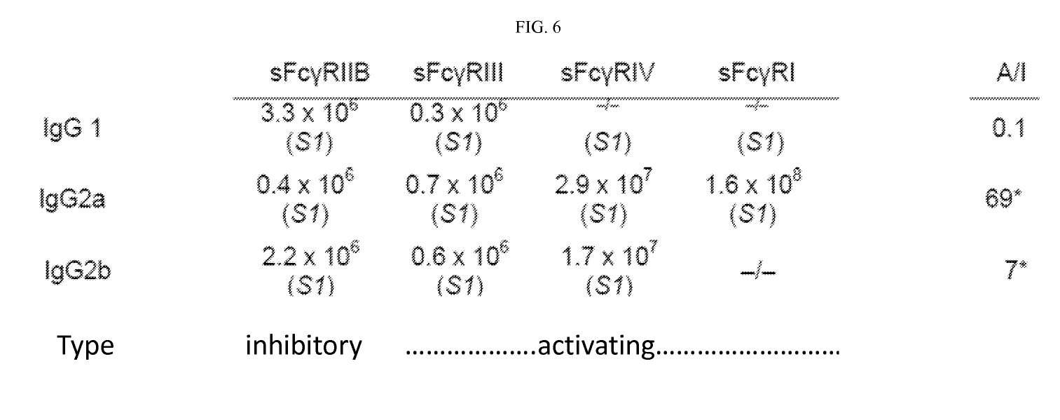

Choice of immunoglobulin isotypes can be an important consideration when developing therapeutic antibodies. Nimmerjahn et al. (Science, 310 (Dec. 2, 2005)) calculated activating/inhibitor ratios (A/I) for model antibodies containing different immunoglobulin isotypes based upon their affinity to inhibitory Fc receptors and activating Fc receptors (see FIG. 6). The ratios are useful for determining whether an antibody may have depleting capability which may be important for predicting in vivo depleting activity of an antibody in humans.

The efficacy of either monotherapy with murine anti-CD137 containing various immunoglobulin isotypes (g1, g1 D254A, g2a, or g2b), or murine anti-PD-1, or combination therapy with murine anti-CD137 containing various immunoglobulin isotypes (g1, g1 D254A, g2a, or g2b) and murine anti-PD-1 antibodies, in mice that were injected with MC38 tumors was tested.

C57BL/6 mice were injected with 2×10⁶ MC38 tumor cells on day 0. On day 8, mice were randomized based on tumor volumes and treated with the indicated mAbs (the mean tumor volume was ˜37 mm3/2). All mice were intraperitoneally injected with 200 ug of each mAb in a total volume of 200 ul in PBS. Mice were injected again on days 11 and 15. Tumor volumes were measured with an electronic caliper twice weekly and recorded as mm³/2. Mice were removed from the study when they were found dead, had ulcerations or had large tumor burdens. The concentrations of the mAbs used are shown in Table 5. The isotype of each mAb is identified after it name (CD137 or PD-1).

TABLE 5

| Treatment | [mg/ml] |
|---|---|
| 1. IgG1 | 3.41 |
| 2. CD137 g1 | 9.3 |
| 3. CD137 g1 D265A | 5.28 |
| 4. CD137 g2b | 3.2 |
| 5. CD137 g2a | 7.8 |
| 6. PD-1 g1 | 6.14 |
| 7. PD-1 + CD137 g1 | As noted above |
| 8. PD-1 + CD137g1 D265A | As noted above |
| 9. PD-1 + CD137 g2b | As noted above |

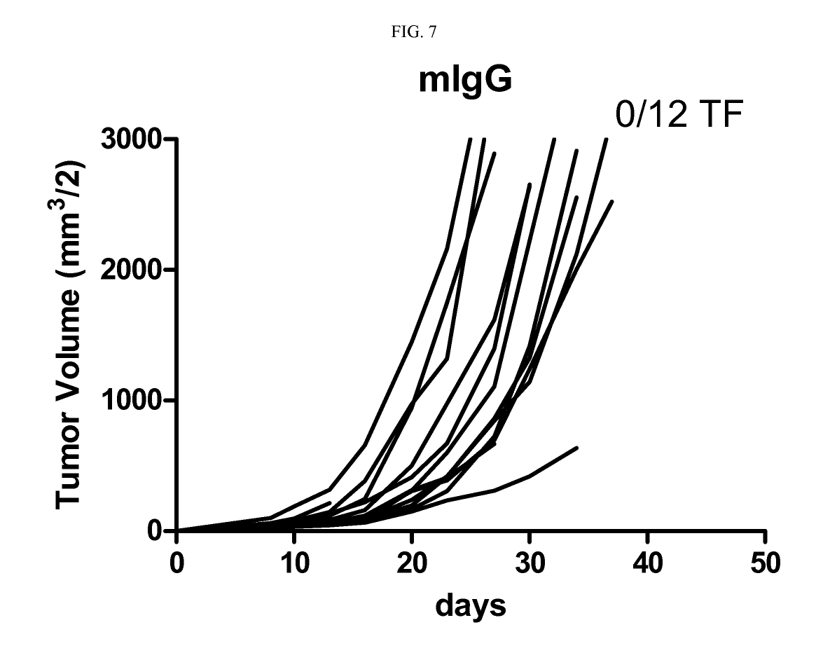

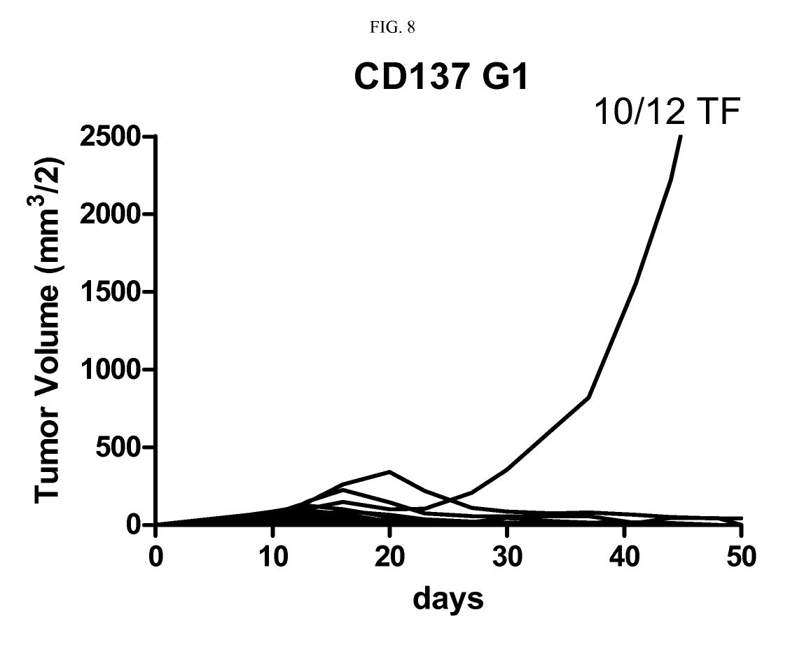

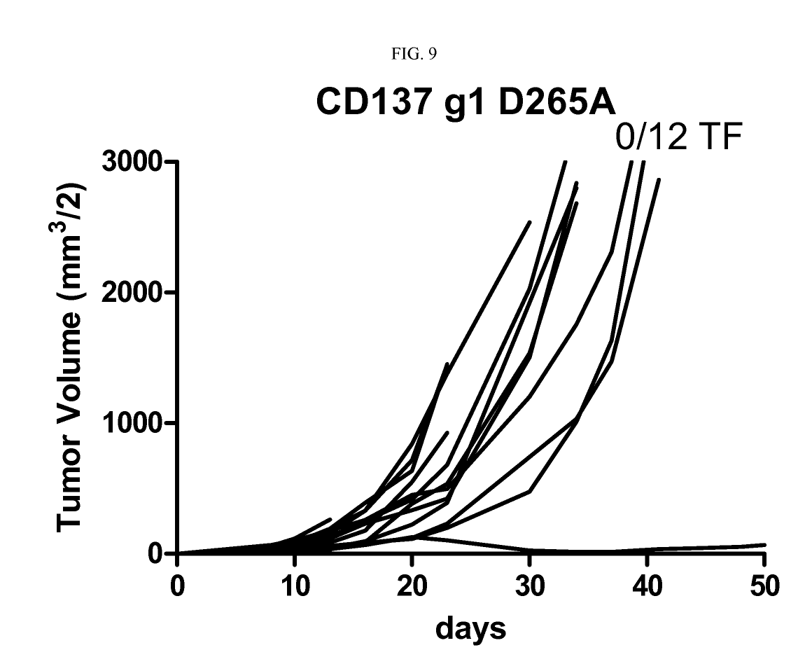

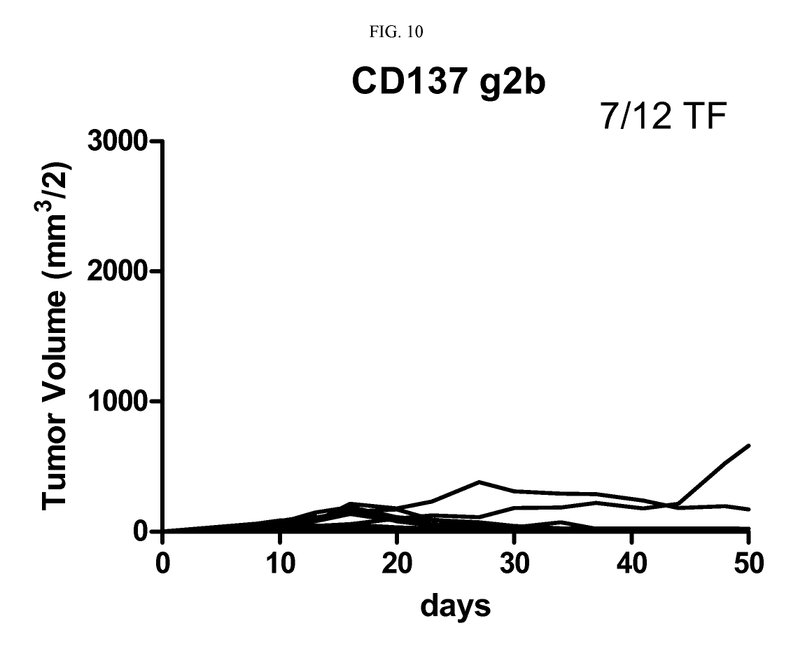

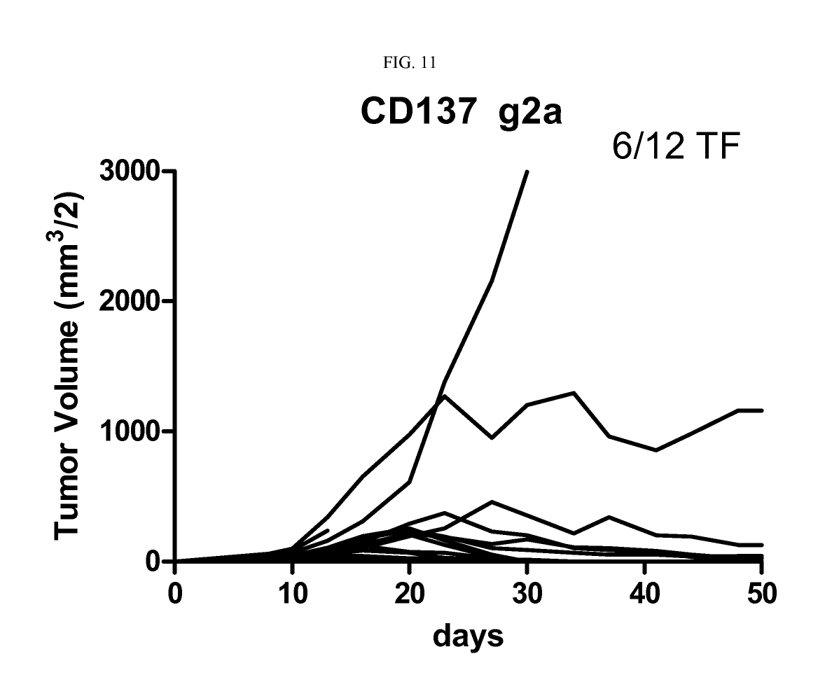

The results are shown in FIGS. 7 thru 20. As shown in FIG. 8, anti-CD137 with the G1 isotype showed potent anti-tumor activity. Generally, the IgG1 isotype has very potent antitumor activity because this isotype can engage Fc receptors which promotes cross-linking and a higher valency interaction with the target, resulting in agonism of T cell responses and increased antitumor responses via ADCC. However, no depletion was anticipated in these experiments due to the low A/I ratio (0.1) that was used in accordance with Nimmerjahn et al. As shown in FIG. 9, little anti-tumor activity was observed with the G1 D265A isotype (a G1 mutation that is unable to engage any Fc receptors and thus is not expected to have effector ADCC function, and thus makes this mutation a good surrogate for the human IgG4 isotype) is consistent with a requirement for Fc receptor engagement for C137 agonism, at least with this CD137 antibody. Antibodies to other CD137 epitopes may function independently of Fc receptor binding. As shown in FIG. 10, the IgG2b isotype is highly effective due to FcR-mediated agonism and depletion of Treg cells, which limit antitumor responses. By comparison, the IgG2a isotype has somewhat weaker antitumor responses than the IgG2b format as shown in FIG. 11. This lower efficacy reflects the greater potential for depletion (A/I=69) of not only Tregs but also CD137+CD8+ T cells, which mediate much of the antitumor activity.

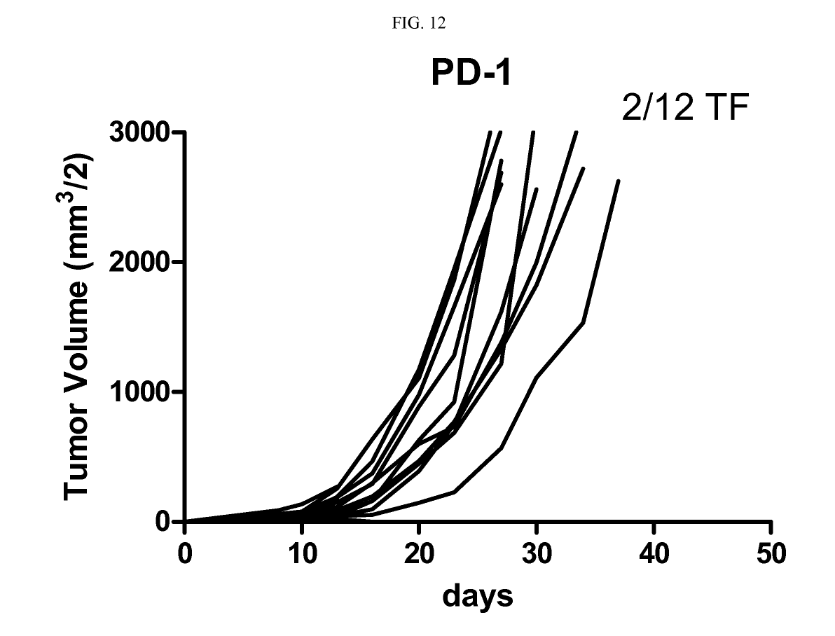

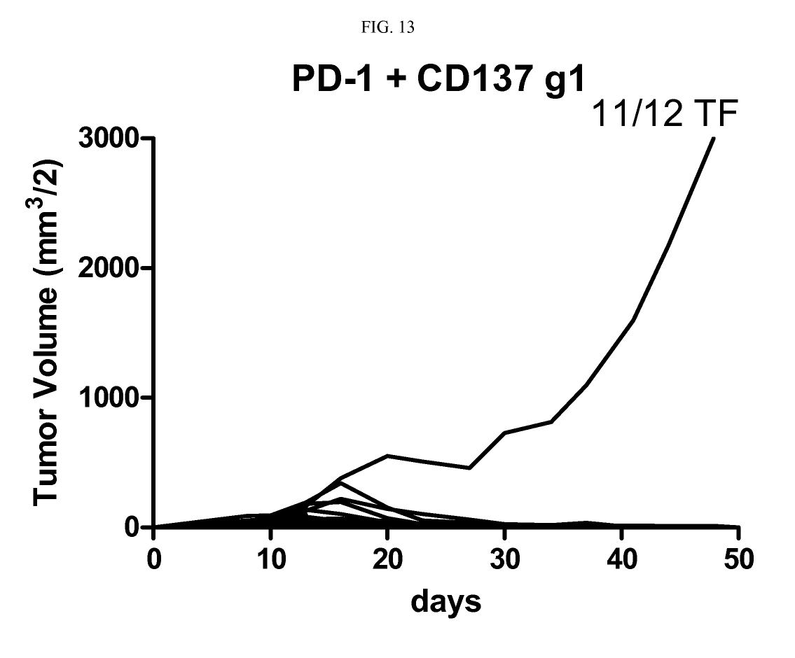

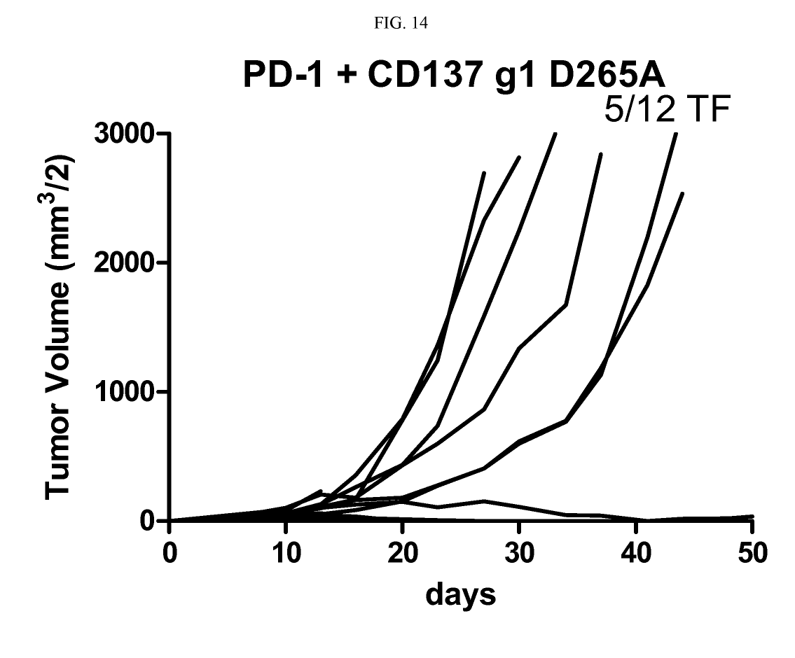

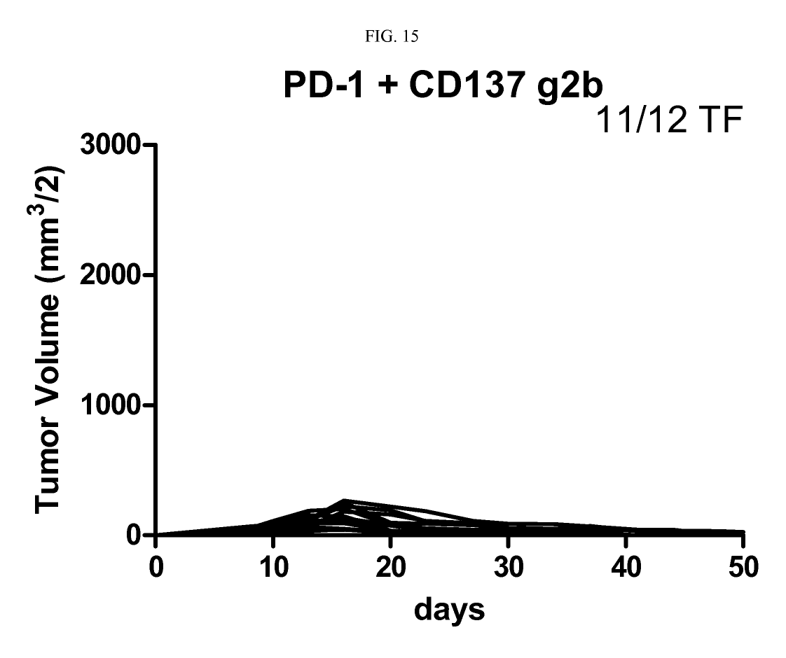

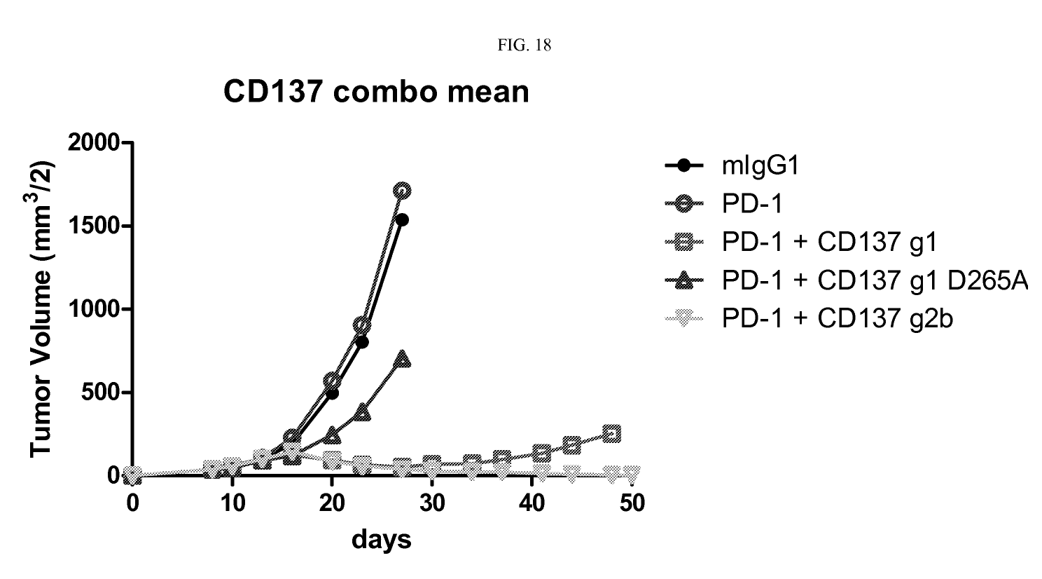

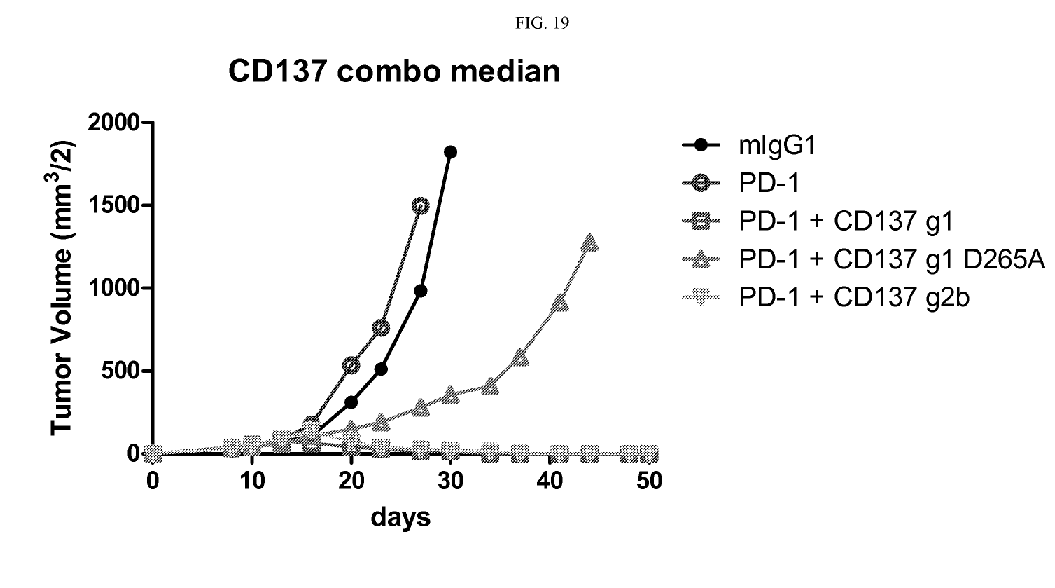

For the mice administered an anti-mouse anti-PD-1, only modest activity as monotherapy was observed. However, when used in combination with the murine anti-CD137 antibodies, significant activity was observed in some of the combinations and less activity in others, depending upon the immunoglobulin isotype that the anti-CD137 antibody contained. Specifically, mice administered both the murine anti-PD-1 and anti-CD137 antibody containing the G1 isotype showed only additive results over mice treated with only the anti-CD137 antibody containing the G1 isotype versus those mice treated with only anti-PD1 (see FIG. 13 relative to FIGS. 12 and 8). But the combination of mouse anti-CD137 containing the Ig D265A mutation and mouse anti-PD-1 showed synergy in terms of greater efficacy than either anti-CD137 containing the G2b D265A mutation or anti-PD1 alone (see FIG. 14 relative to FIGS. 12 and 9). In addition, the combination of mouse anti-CD137 containing the G2b isotype and mouse anti-PD-1 showed synergy in terms of greater efficacy than either anti-CD137 containing the G2b isotype or anti-PD1 alone (see FIG. 15 relative to FIGS. 12 and 10). Results showing the enhanced activity of the combination and the anti-PD1 antibody alone in terms of both the mean and median reduction in tumor volumes (see FIGS. 18 and 19) was also observed with the combination of anti-PD1 and anti-CD137 antibodies containing the g2b isotype achieved the best response, followed by g1.

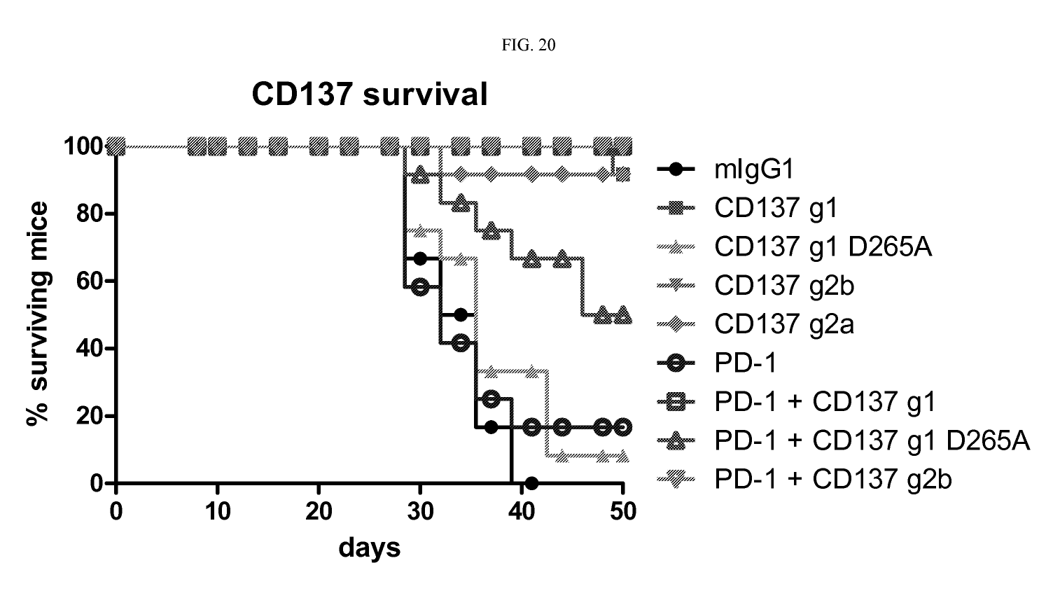

The enhanced activity of the murine anti-CD137 isotype antibodies in combination with the anti-PD1 antibody was also observed when the overall survival of the mice was evaluated (see FIG. 20) with the combination of anti-PD1 and anti-CD137 antibodies containing the g2b and g1 isotypes achieving the best responses.

These preliminary data suggest that the combination of an anti-PD1 antibody with anti-CD137 antibodies, particularly when the anti-CD137 antibodies contain a g2b or g1 isotypes, leads to increased efficacy in treating tumors. In addition, the enhanced synergistic effect would likely be very effective when the anti-CD137 antibody binds to an epitope of CD137 that promotes sufficient Fc receptor engagement to permit CD137 agonism.

These results provide further pre-clinical data in relevant murine animal models to support the potential benefit of combining anti-CD137 (e.g., urelumab) and anti-PD-1 (e.g., nivolumab) antibodies in human clinical trials.

### BRIEF DESCRIPTION OF THE SEQUENCE LISTING

Incorporated herein by reference in its entirety is a Sequence Listing entitled, “12380WOPCT_ST25”, comprising SEQ ID NO:1 through SEQ ID NO:20, which includes the nucleic acid and/or amino acid sequences disclosed herein. The Sequence Listing has been submitted herewith in ASCII text format via EFS. The Sequence Listing was first created on Aug. 12, 2015, and is 18 KB in size.

### SEQUENCE SUMMARY

| SEQ | |
|---|---|
| ID | |
| NO: | SEQUENCE |
| 1 | Heavy Chain Amino Acid Sequence |
| | Anti-CD137 mAb (BMS-663513) |
| | (variable region underlined; constant region bold) |
| | (residues 20-467 of SEQ ID NO: 3 from U.S. Pat. No. 7,288,638) |
| | QVQLQQWGAGLLKPSETLSLTCAVYGGSFSGYYWSWIRQSPEKGLEWIGEINHG |
| | GYVTYNPSLESRVTISVDTSKNQFSLKLSSVTAADTAVYYCARDYGPGNYDWYF |
| | DLWGRGTLVTVSSASTKGPSVFPLAPCSRSTSESTAALGCLVKDYFPEPVTVSW |
| | NSGALTSGVHTFPAVLQSSGLYSLSSVVTVPSSSLGTKTYTCNVDHKPSNTKVD |
| | KRVESKYGPPCPPCPAPEFLGGPSVFLFPPKPKDTLMISRTPEVTCVVVDVSQE |
| | DPEVQFNWYVDGVEVHNAKTKPREEQFNSTYRVVSVLTVLHQDWLNGKEYKCKV |
| | SNKGLPSSIEKTISKAKGQPREPQVYTLPPSQEEMTKNQVSLTCLVKGFYPSDI |
| | AVEWESNGQPENNYKTTPPVLDSDGSFFLYSRLTVDKSRWQEGNVFSCSVMHEA |
| | LHNHYTQKSLSLSLGK |
| 2 | Light Chain Amino Acid Sequence |
| | Anti-CD137 mAb (BMS-663513) |
| | (variable region underlined; constant region bold) |
| | (residues 21-236 of SEQ ID NO: 6 from U.S. Pat. No. 7,288,638) |
| | EIVLTQSPATLSLSPGERATLSCRASQSVSSYLAWYQQKPGQAPRLLIYDASNRA |
| | TGIPARFSGSGSGTDFTLTISSLEPEDFAVYYCQQRSNWPPALTFGGGTKVEIKR |
| | TVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 3 | Heavy Chain Variable Region (VH) Amino Acid Sequence |
| | Anti-CD137 mAb (BMS-663513) |
| | (residues 20-140 of SEQ ID NO: 3 from U.S. Pat. No. 7,288,638) |
| | QVQLQQWGAGLLKPSETLSLTCAVYGGSFSGYYWSWIRQSPEKGLEWIGEINHGG |
| | YVTYNPSLESRVTISVDTSKNQFSLKLSSVTAADTAVYYCARDYGPGNYDWYFDL |
| | WGRGTLVTVSS |
| 4 | Light Chain Variable Region (VL) Amino Acid Sequence |
| | Anti-CD137 mAb (BMS-663513) |
| | (residues 21-129 of SEQ ID NO: 6 from U.S. Pat. No. 7,288,638) |
| | EIVLTQSPATLSLSPGERATLSCRASQSVSSYLAWYQQKPGQAPRLLIYDASNRA |
| | TGIPARFSGSGSGTDFTLTISSLEPEDFAVYYCQQRSNWPPALTFGGGTKVEIK |
| 5 | Heavy Chain CDR1 Amino Acid Sequence |
| | Anti-CD137 mAb (BMS-663513) |
| | (residues 50-54 of SEQ ID NO: 3 from U.S. Pat. No. 7,288,638) |
| | GYYWS |
| 6 | Heavy Chain CDR2 Amino Acid Sequence |
| | Anti-CD137 mAb (BMS-663513) |
| | (residues 69-84 of SEQ ID NO: 3 from U.S. Pat. No. 7,288,638) |
| | EINHGGYVTYNPSLES |
| 7 | Heavy Chain CDR3 Amino Acid Sequence |
| | Anti-CD137 mAb (BMS-663513) |
| | (residues 117-129 of SEQ ID NO: 3 from U.S. Pat. No. 7,288,638) |
| | DYGPGNYDWYFDL |
| 8 | Light Chain CDR1 Amino Acid Sequence |
| | Anti-CD137 mAb (BMS-663513) |
| | (residues 44-54 of SEQ ID NO: 6 from U.S. Pat. No. 7,288,638) |
| | RASQSVSSYLA |
| 9 | Light Chain CDR2 Amino Acid Sequence |
| | Anti-CD137 mAb (BMS-663513) |
| | (residues 70-76 of SEQ ID NO: 6 from U.S. Pat. No. 7,288,638) |
| | DASNRAT |
| 10 | Light Chain CDR3 Amino Acid Sequence |
| | Anti-CD137 mAb (BMS-663513) |
| | (residues 109-119 of SEQ ID NO: 6 from U.S. Pat. No. 7,288,638) |
| | QQRSNWPPALT |
| 11 | Heavy Chain Amino Acid Sequence |
| | Anti-PD-1 mAb (BMS936558; 5C4 in WO 2006/121168) |
| | (variable region underlined; constant region bold) |
| | QVQLVESGGGVVQPGRSLRLDCKASGITFSNSGMHWVRQAPGKGLEWVAVIWYDG |
| | SKRYYADSVKGRFTISRDNSKNTLFLQMNSLRAEDTAVYYCATNDDYWGQGTLVT |
| | VSSASTKGPSVFPLAPCSRSTSESTAALGCLVKDYFPEPVTVSWNSGALTSGVHT |
| | FPAVLQSSGLYSLSSVVTVPSSSLGTKTYTCNVDHKPSNTKVDKRVESKYGPPCP |
| | PCPAPEFLGGPSVFLFPPKPKDTLMISRTPEVTCVVVDVSQEDPEVQFNWYVDGV |
| | EVHNAKTKPREEQFNSTYRVVSVLTVLHQDWLNGKEYKCKVSNKGLPSSIEKTIS |
| | KAKGQPREPQVYTLPPSQEEMTKNQVSLTCLVKGFYPSDIAVEWESNGQPENNYK |
| | TTPPVLDSDGSFFLYSRLTVDKSRWQEGNVFSCSVMHEALHNHYTQKSLSLSLGK |
| 12 | Light Chain Amino Acid Sequence |
| | Anti-PD-1 mAb (BMS936558; 5C4 in WO 2006/121168) |
| | (variable region underlined; constant region bold) |
| | EIVLTQSPATLSLSPGERATLSCRASQSVSSYLAWYQQKPGQAPRLLIYDASNRA |
| | TGIPARFSGSGSGTDFTLTISSLEPEDFAVYYCQQSSNWPRTFGQGTKVEIKRTV |
| | AAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 13 | Heavy Chain Variable Region (VH) Amino Acid Sequence |
| | Anti-PD-1 mAb (BMS936558; 5C4 in WO 2006/121168) |
| | (SEQ ID NO: 4 from WO 2006/121168) |
| | QVQLVESGGGVVQPGRSLRLDCKASGITFSNSGMHWVRQAPGKGLEWVAVIWYDG |
| | SKRYYADSVKGRFTISRDNSKNTLFLQMNSLRAEDTAVYYCATNDDYWGQGTLVT |
| | VSS |
| 14 | Light Chain Variable Region (VL) Amino Acid Sequence |
| | Anti-PD-1 mAb (BMS936558; 5C4 in WO 2006/121168) |
| | (SEQ ID NO: 11 from WO 2006/121168) |
| | EIVLTQSPATLSLSPGERATLSCRASQSVSSYLAWYQQKPGQAPRLLIYDASNRA |
| | TGIPARFSGSGSGTDFTLTISSLEPEDFAVYYCQQSSNWPRTFGQGTKVEIK |
| 15 | Heavy Chain CDR1 Amino Acid Sequence |
| | Anti-PD-1 mAb (BM5936558; 5C4 in WO 2006/121168) |
| | (SEQ ID NO: 18 from WO 2006/121168) |
| | NSGMH |
| 16 | Heavy Chain CDR2 Amino Acid Sequence |
| | Anti-PD-1 mAb (BMS936558; 5C4 in WO 2006/121168) |
| | (SEQ ID NO: 25 from WO 2006/121168) |
| | VIWYDGSKRYYADSVKG |
| 17 | Heavy Chain CDR3 Amino Acid Sequence |
| | Anti-PD-1 mAb (BMS936558; 5C4 in WO 2006/121168) |
| | (SEQ ID NO: 32 from WO 2006/121168) |
| | NDDY |
| 18 | Light Chain CDR1 Amino Acid Sequence |
| | Anti-PD-1 mAb (BMS936558; 5C4 in WO 2006/121168) |
| | (SEQ ID NO: 39 from WO 2006/121168) |
| | RASQSVSSYLA |
| 19 | Light Chain CDR2 Amino Acid Sequence |
| | Anti-PD-1 mAb (BMS936558; 5C4 in WO 2006/121168) |
| | (SEQ ID NO: 46 from WO 2006/121168) |
| | DASNRAT |
| 20 | Light Chain CDR3 Amino Acid Sequence |
| | Anti-PD-1 mAb (BMS936558; 5C4 in WO 2006/121168) |
| | (SEQ ID NO: 53 from WO 2006/121168) |
| | QQSSNWPRT |

What is claimed is:

1. A method for treating a subject afflicted with a cancer, comprising administering to the subject a combination of therapeutically effective amounts of:
(a) a monoclonal antibody or an antigen-binding portion thereof that binds specifically to a Programmed Death-1 (PD-1) receptor; and
(b) a monoclonal antibody or an antigen-binding portion thereof that binds specifically to CD137.

2. The method of claim 1, wherein the cancer is a solid tumor.

3. The method of claim 2, wherein the solid tumor is selected from melanoma, prostate cancer, non-small cell lung cancer, colorectal cancer, head and neck squamous cell carcinoma, renal cell carcinoma, gastric carcinoma, glioblastoma, and Non-Hodgkin's Lymphoma.

4. The method of claim 1, wherein the cancer is a B cell lymphoma.

5. The method of claim 4, wherein the cancer is a B-cell non-Hodgkin's lymphoma.

6. The method of claim 1, wherein the anti-PD-1 antibody cross-competes with nivolumab for binding to human PD-1.

7. The method of claim 1, wherein the anti-PD-1 antibody is a chimeric, humanized or human monoclonal antibody or a portion thereof.

8. The method of any one of claims 1-7, wherein the anti-PD-1 antibody comprises a heavy chain constant region which is of a human IgG1, IgG2, IgG4 isotype, or variant thereof.

9. The method of claim 1, wherein the anti-PD-1 antibody is nivolumab.

10. The method of claim 1, wherein the anti-PD-1 antibody is administered at a dose of 1 or 3 mg/kg body weight once every 2 weeks.

11. The method of claim 1, wherein the anti-CD137 antibody cross-competes with urelumab for binding to human CD137.

12. The method of claim 12, wherein the anti-CD137 antibody is a chimeric, humanized or human monoclonal antibody or a portion thereof.

13. The method of claim 1, wherein the anti-CD137 antibody comprises a heavy chain constant region which is of a human IgG1, IgG2, or IgG4 isotype.

14. The method of claim 1, wherein the anti-CD137 antibody is urelumab.

15. The method of claim 1, wherein the anti-CD137 antibody is administered at a dose of 3 or 8 mg once every 4 weeks.

16. The method of claim 1, wherein the method comprises at least one treatment cycle of eight weeks.

17. The method of claim 16, wherein the anti-PD-1 antibody is administered on Days 1, 15, 29, and 43 of each cycle.

18. The method of claim 16, wherein the anti-CD137 antibody is administered on Days 1 and 29 of each cycle or Day 1 of each cycle.

19. A kit for treating a subject afflicted with a cancer, comprising:
(a) a dosage ranging from 0.1 to 10 mg/kg body weight of an anti-PD-1 antibody or an antigen-binding portion thereof;
(b) a dosage ranging from 1 to 10 mg of an anti-CD137 antibody or an antigen-binding portion thereof; and
(c) instructions for using the anti-PD-1 antibody and the anti-CD137 antibody in the method of any of claims 1-38.

20. The method according to claim 1, wherein the anti-CD137 antibody binds to an CD137 epitope that either crosslinking and/or promotes Fc receptor engagement and results in CD137 agonism.


SEQUENCE LISTING (20 sequences)

SEQ ID NO: 1 - protein, 448 aa, Homo sapiens
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 216 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 121 aa, Homo sapiens [sequence omitted]

SEQ ID NO: 4 - protein, 109 aa, Homo sapiens [sequence omitted]

SEQ ID NO: 5 - protein, 5 aa, Homo sapiens
GYYWS

SEQ ID NO: 6 - protein, 16 aa, Homo sapiens
EINHGGYVTYNPSLES

SEQ ID NO: 7 - protein, 13 aa, Homo sapiens
DYGPGNYDWYFDL

SEQ ID NO: 8 - protein, 11 aa, Homo sapiens
RASQSVSSYLA

SEQ ID NO: 9 - protein, 7 aa, Homo sapiens
DASNRAT

SEQ ID NO: 10 - protein, 11 aa, Homo sapiens
QQRSNWPPALT

SEQ ID NO: 11 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 113 aa, Homo sapiens [sequence omitted]

SEQ ID NO: 14 - protein, 107 aa, Homo sapiens [sequence omitted]

SEQ ID NO: 15 - protein, 5 aa, Homo sapiens
NSGMH

SEQ ID NO: 16 - protein, 17 aa, Homo sapiens
VIWYDGSKRYYADSVKG

SEQ ID NO: 17 - protein, 4 aa, Homo sapiens
NDDY

SEQ ID NO: 18 - protein, 11 aa, Homo sapiens
RASQSVSSYLA

SEQ ID NO: 19 - protein, 7 aa, Homo sapiens
DASNRAT

SEQ ID NO: 20 - protein, 9 aa, Homo sapiens
QQSSNWPRT